(19) United States
(12) Patent Application Publication
(10) Pub. No.: US 2004/0034357 A1
(43) Pub. Date: Feb. 19, 2004

(54) Controlled release implantable devices

(75) Inventors: Richard M. Beane, Hingham, MA (US); Carol L. Miller-Graziano, Holden, MA (US); Steve Ek, Bolton, MA (US); George Sikora, Mansfield, MA (US); Allison Niemann, Burlingame, CA (US); Demetrius Litwin, Westboro, MA (US)

Correspondence Address: FISH & RICHARDSON PC, 225 FRANKLIN ST, BOSTON, MA, 02110, US

(73) Assignees: UNIVERSITY OF MASSACHUSETTS, a Massachusetts corporation; SMITH & NEPHEW, INC., a Delaware corporation

(21) Appl. No.: 10/421,103

(22) Filed: Apr. 23, 2003

Related U.S. Application Data
(63) Continuation of application No. 09/632,133, filed on Aug. 3, 2000, now abandoned.
(60) Provisional application No. 60/146,909, filed on Aug. 3, 1999.

(51) Int. Cl. A61B 17/56

(52) U.S. Cl. 606/73

(57) ABSTRACT

Implantable devices that deliver a drug to a target location are disclosed. In general, the devices include a portion that engages tissue near the target location and a section with an internal cavity that houses the drug. The drug can be incorporated into a controlled release agent, such as drug-polymer microspheres, which steadily or intermittently release the drug. Alternatively, instead of defining an internal cavity, the section can be formed from a material that includes the controlled release agent. The devices can be used, e.g., to deliver down-regulatory cytokines to a site of inflammation.

### CROSS-REFERENCE TO RELATED APPLICATIONS

This application claims priority to and incorporates by reference U.S. patent application Ser. No. 09/632,133, filed Aug. 3, 2000, and U.S. Provisional Patent Application No. 60/146,909, filed Aug. 3, 1999.

### FIELD OF THE INVENTION

The invention relates to implantable devices for delivering drugs to a desired location within a body.

### BACKGROUND OF THE INVENTION

Drugs can be delivered systemically, e.g., by oral ingestion, or can be delivered locally directly to a site of disease. Some drugs are most effective if delivered repeatedly, over a period of time, or delivered steadily, e.g., using an implantable device.

### SUMMARY OF THE INVENTION

The invention relates to new implantable devices specially designed to deliver drugs to desired locations adjacent to unique target sites in bone, cartilage, ligaments, muscle, and other internal body tissues and structures, and to provide a controlled release of a wide variety of drugs. In some embodiments, the devices also perform a mechanical function, e.g., attaching tissue to a support structure, such as bone.

In general, in one aspect, the invention features an implantable device for attaching tissue to a support structure inside a body and for delivering a drug to a target location near the support structure. The device includes a first portion that engages the tissue, and a second portion that engages the support structure. A section of the device defines an internal cavity that has a size and shape for containing a controlled release agent that includes the drug. Alternatively, instead of defining an internal cavity, at least a portion of the section can be formed from a material that comprises the controlled release agent that includes the drug. The section can be part of the first portion, part of the second portion, or a separate section connected to either the first portion, the second portion, or both.

Embodiments of this aspect of the invention may include one or more of the following features. The device can include the controlled release agent. The controlled release agent can be a mixture of a polymer and the drug, e.g., microspheres of the polymer containing the drug. The controlled release agent can be configured to release the drug for a period greater than, e.g., two days or five weeks. The agent can also be configured to release the drug intermittently over a period of time.

The drug can be, e.g., a down-regulatory cytokine, such as interleukin-10, a pain killer, such as lidocaine, platelet derived growth factor, an antibiotic, a hormone, a prostaglandin, a protein, a peptide sequence, or a nucleic acid. The polymer can be, e.g., a polyanhydride, a polylactide, a polyglycolide, a polylactic acid, a polyglycolic acid, a polyorthoester, a polyorthocarbonate, a polyacetal, a polymer derived from alpha hydroxycarboxylic acids and lactones, a polymer derived from condensation of divinyl ethers and polyols, an e-caprolactbne polymer, ethylene vinyl acetate copolymer, and other co-polymers of the above listed polymers, such as 50:50 poly(DL-lactide-co-glycolide).

The second portion of the device can be configured to penetrate the tissue, e.g., with a pointed end. The section can be degradable by bodily fluids. In addition, the section can have an aperture that exposes the interior cavity to bodily fluids when the device is implanted in the body. A membrane permeable to bodily fluids and to the drug only when the drug is dissolved or suspended in bodily fluids can cover the aperture. The section can also include the first and/or the second portion.

The tissue can be soft tissue or bony tissue, and the support structure can include bone.

In another aspect, the invention features an implantable device for delivering a drug to a desired location inside a body. The device includes a rigid exterior that has a tapered end for penetrating tissue within the body, and a projection for engaging tissue within the body. The device also includes an internal cavity in fluid communication with the rigid exterior. The cavity has a size and shape for containing a controlled release agent that includes the drug.

Embodiments of this aspect of the invention may include one or more of the following features. The rigid exterior has a pointed, arrow-shaped head that includes both the tapered end and the projection. The arrow-shaped head can have a shaft and two projections, each projection having a first pointed end and a second end connected to the shaft. The first ends are movable between a first position flush with the shaft, and a second position displaced away from the shaft.

In another aspect, the invention features an implantable staple for delivering a drug to a desired location within a body. The staple includes at least two prongs that penetrate and engage tissue, and a shaft connecting the two prongs. The shaft has an internal cavity that has a size and shape for containing a controlled release agent that includes the drug. The shaft can include a material degradable by bodily fluids.

The invention also features an implantable device for delivering a drug to a target location. The device includes an elongated rod curved in a generally helical shape. The helical shape tapers to a point that in use penetrates soft tissue, and the helical shape forms a conical interior space configured to contain a solid controlled release agent that includes the drug.

In another aspect, the invention features an implantable device for delivering drug to a target location. The device has a body that includes a controlled release agent that includes the drug, and has a through-hole for passage of a guide wire therethrough.

Embodiments of this aspect of the invention may include one or more of the following features. The body includes a shell that surrounds the controlled release agent. The shell has a head and a shaft, and defines a bore. The bore contains a medicament core that includes the controlled release agent, and defines the through-hole. The device can further include a tissue engaging projection connected to the shaft.

Furthermore, the invention includes an implantable suture anchor for delivering a drug to a desired location in a body. The suture anchor includes an exterior shell that defines a hole for passage of a suture therethrough, and an internal cavity within the shell in fluid communication with the hole. The cavity has a size and shape for containing a controlled release agent that includes the drug. The anchor also includes a membrane covering the hole to retain the agent within the cavity. The membrane is permeable to bodily fluids and to the drug when the drug is dissolved or suspended in bodily fluids. The exterior shell of the anchor can include a material that is degradable by bodily fluids.

In another aspect, the invention features an implantable bone screw that has a rigid, threaded shaft for penetrating bone, and an internal cavity within the shaft. The internal cavity has a size and shape for containing a controlled release agent that includes a drug.

Embodiments of this aspect of the invention may include one or more of the following features. The bone screw can include the controlled release agent, and the controlled release agent can be a mixture of the drug and a polymer, the mixture formulated to provide controlled release of the drug. The shaft of the bone screw can define an aperture that opens into the cavity. The aperture can be covered by a membrane that is permeable to bodily fluids and the drug only when the drug is dissolved or suspended in bodily fluids. The aperture can be located on a cylindrical threaded wall of the shaft. In addition, the shaft can define a plurality of apertures that open into the cavity.

The invention also features an implantable anchor for delivering a drug to a desired location in a body. The anchor includes a laterally expandable shaft, a plurality of prongs connected to a distal end of the shaft. The prongs are movable between a contracted position and an expanded position, and form an interior hollow space configured to contain a controlled release agent that includes the drug.

In another aspect, the invention features an implantable suture anchor for delivering a drug to a desired location within a body. The suture anchor includes a pellet formed from a mixture of the drug and a polymer, where the mixture is formulated for controlled release of the drug, and a suture passing through the pellet for implanting the pellet within the body.

Further, the invention includes a splaying implantable device for delivering a drug to a desired location within a body. The device includes a pellet that comprises a controlled release agent which includes the drug, and a splaying anchor connected to the pellet. The anchor has at least two prongs that in use penetrate soft tissue. A distance separating the two prongs increases when the prongs are inserted into the tissue.

In another aspect, the invention features an implantable staple for delivering a drug to a desired location within a body. The staple is formed from a material comprising a mixture of the drug and a polymer, where the mixture is formulated for controlled release of the drug. The staple includes at least two prongs for penetrating soft tissue and a shaft connecting the two prongs.

The invention also features an implantable device for delivering a drug to a desired location inside a body formed from, e.g., woven or braided threads. The device includes a section formed from a sheet of one or more polymer threads molded to form the section. The section of the device defines an internal cavity that has a size and shape for containing a controlled release agent that includes the drug.

Embodiments of this aspect of the invention may include one or more of the following features. The one or more threads can be woven to form the sheet, or compressed to form a mesh sheet. The device can include the controlled release agent, and the controlled release agent can be a cylindrical pellet that includes the drug.

In another embodiment, the invention includes a method of attaching tissue to a support structure and delivering a drug to a target location inside a body. The method includes: (a) obtaining one of the implantable devices described above; and (b) implanting the device within the body by engaging the second portion with the support structure, and the first portion with soft tissue, such that the agent releases the drug to the desired location over time. In this method, the device can be made from a material degradable by bodily fluids.

In another aspect, the invention features a method of treating inflammatory disease. The method includes obtaining an implantable device that in use contains a down-regulatory cytokine, e.g., interleukin-10, and implanting the device in proximity to a site of inflammation in the body. The implantable device then releases the down-regulatory cytokine to the site of inflammation.

This aspect of the invention may include one or more of the following features. The implantable device can contain a sustained release formulation that includes the down-regulatory cytokine, such that the device releases the down regulatory cytokine steadily over a period of time greater than, e.g., two days, greater than five days, or greater than five weeks. The sustained release formulation can be a mixture of the drug and a polymer, e.g., microspheres that include the drug and the polymer.

Embodiments of the invention may include one or more of the following advantages. By engaging an internal body structure in proximity to a target area, the implantable devices focus delivery of the drug to a target area. The devices are specially designed to remain engaged with internal body structures near the target site, allowing controlled, e.g., continuous, sustained or intermittent, release of a drug to a target site.

The rigid exteriors of certain embodiments of the invention protect the controlled-release agent, avoiding rupture of the agent and promoting controlled release of the drug. The devices formed entirely from a drug-polymer mixture have the advantage of being formed from a single, unitary piece.

The devices allow controlled, e.g., sustained, release of a drug to a target site over periods of, e.g., several hours, one or more days, several weeks, months, or longer. Other devices control the release of a drug to provide one or more doses per day for several days to weeks or months.

Many of the devices perform a second function in addition to sustained release of a drug. For example, the tissue staples and T-fixes described below can be used for wound closure, and the bone screws and soft tissue tacks can be used, e.g., in ligament replacement surgeries.

The microsphere conglomerates of certain embodiments are relatively simple to manufacture and promote steady release of specific amounts of a drug when exposed to bodily fluids.

The devices obviate the need for systemic delivery of drugs, or repeated injections with needles to a target area. For the embodiments relating to delivery of down-regulatory cytokines such as IL-10, targeting therapy to a site of inflammation is particularly desirable, since IL-10 has a short lifespan, and since systemic delivery of IL-10 could potentially interfere with proper functioning of the immune system.

As used herein, a “body” is a human or animal body, unless specifically described as one or the other.

“Bodily fluids” are liquids within a body which may or may not include cells. For example, blood, digestive fluids, lymphatic fluids, plasma, and waste fluids are all “bodily fluids.”,

“Soft tissue” is any tissue found in a body that is less rigid than bone. For example, muscle, tendons and ligaments, and organs are all made from “soft tissue.”

A “support structure” is a structure within the body that has sufficient structural integrity to support an attached implantable device. Bone is an example of a support structure. Rigd artificial structures implanted in the body, such as plastic or metal plates or screws, can also serve as support structures.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and are not intended to be limiting.

Other features and advantages of the invention will be apparent from the following detailed description, and from the claims.

### BRIEF DESCRIPTION OF THE DRAWINGS

FIG. 1A is a perspective view of a drug-polymer T-fix with a splaying anchor.

FIG. 1B illustrates the T-fix of FIG. 1A implanted into tissue.

FIG. 2 is a perspective view of an alternative-drug-polymer T-fix having a suture passed therethrough rather than a splaying anchor.

FIG. 3A is a perspective view of an implantable drug-polymer plug.

FIG. 3B is a cross-sectional view of the plug of FIG. 3A.

FIG. 4 is a perspective view of an implantable drug-polymer staple.

FIG. 5A is a perspective, diagrammatic view of a drug delivery T-fix having a rigid exterior.

FIG. 5B is a schematic illustrating implantation of the T-fix of FIG. 5A into a knee.

FIG. 6A is a perspective view of a drug delivery bone screw.

FIG. 6B is a schematic illustrating implantation of the bone screw of FIG. 6A into a knee.

FIG. 7A is a perspective view of an apertured drug delivery bone screw and a drug-polymer pellet for insertion into the bone screw.

FIG. 7B is a sectional view of the bone screw and pellet of FIG. 7A.

FIG. 8A is a perspective view of a drug delivery plug and delivery probe, shown separated.

FIG. 8B is a perspective view of the plug and probe of FIG. 8A, shown attached to each other.

FIGS. 8C-8F illustrate implantation of the plug of FIG. 8A using the probe of FIG. 8A.

FIG. 9A is a perspective view of a drug delivery soft tissue tacker and a drug-polymer pellet.

FIG. 9B is a perspective view of a drug delivery soft tissue tacker made from a woven polymer fabric, and the drug-polymer pellet of FIG. 9A.

FIG. 10 is a perspective view of a drug delivery soft tissue staple and a drug-polymer pellet.

FIG. 11 is a perspective view of a drug delivery helical anchor and a drug-polymer pellet.

FIG. 12A is an exploded view of a drug delivery implantable disk.

FIG. 12B is a perspective view of an apparatus for implanting the disk of FIG. 12A.

FIG. 13A is a perspective view of a drug delivery soft tissue tack with a drug-polymer medicament core.

FIG. 13B is a sectional view of the tack of FIG. 13A.

FIG. 14A is a perspective view of a expandable drug delivery anchor with a plug partially inserted therein.

FIG. 14B is a perspective view of the anchor of FIG. 14A with the plug fully inserted.

FIG. 14C is a sectional view of the anchor and plug of FIG. 14A.

FIG. 15 is a partially perspective, partially sectional view of a microsphere.

FIG. 16A is a sectional view of a sectored drug-polymer pellet configured for intermittent release of the drug.

FIG. 16B is a sectional, end view of a layered drug-polymer pellet configured for intermittent release of the drug.

FIG. 17 is a diagrammatic, sectional view of a mold for compressing a drug-polymer powder into a pellet.

### DETAILED DESCRIPTION

Embodiments of the invention relate to a family of implantable devices for delivering a drug to a target site. Each device includes a drug-polymer mixture formulated for controlled release of the drug, and a portion constructed to engage or affix to one or more specific internal body structures, such as soft tissue or bone. As described below, the devices have a variety of shapes and sizes.

The devices can be used to treat a variety of localized conditions. For example, as described in the Examples provided below, inflammatory disease can be treated directly at a site of inflammation by implanting a device containing a mixture of a polymer and interleukin-10 (IL-10)

### Implantable Devices

The implantable devices described herein include a mixture of a drug and a biodegradable polymer, and a portion for engaging or affixing the device to internal body tissue, such as muscle tissue, or a support structure, such as a bone, for an extended period of time without significant shifting or drifting from the target site. As described below, the drug-polymer mixture is formulated to release the drug in a controlled fashion, e.g., steadily or in specified pulses, over an extended period of time.

The devices can generally be divided into two groups: those having at least a portion constructed from the drug-polymer mixture, and those which include an exterior and a cavity for containing the drug-polymer mixture. The structure and operation of representative shaped implantable devices, the structure and operation of representative cavity containing, or “hollow” implantable devices, and suitable materials and methods of manufacture for both groups of devices are described below.

### Shaped Implantable Devices

The shaped implantable devices are constructed from a drug-polymer mixture molded into a desired shape, or include at least a portion made of such drug-polymer mixtures.

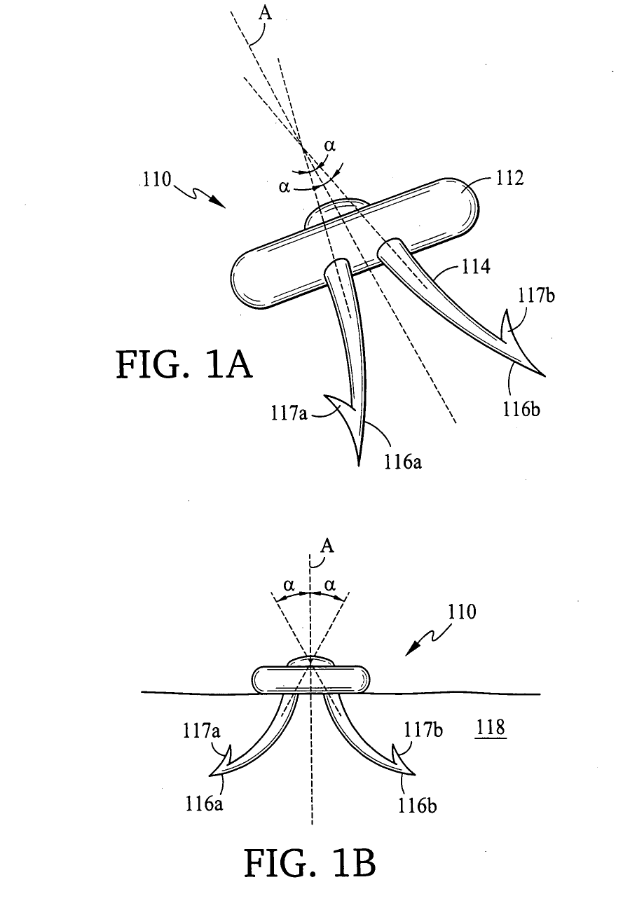

FIG. 1A illustrates a T-fix 110. T-fix 110 has a pellet 112 formed from a drug-polymer mixture, and a splaying anchor 114 formed from a flexible, absorbable polymer, such as polyglycolic acid or polylactic glycolic acid. Anchor 114 has two flexible prongs, 116a, 116b, for penetrating soft tissue near a target site. Each prong 116a, 116b forms an angle α with a longitudinal axis A of T-fix 110. When T-fix 110 is at rest, outside of tissue, angle α is, e.g., about 10°. Each prong 116a, 116b also includes a pointed barb 117a, 117b.

Referring to FIG. 1B, T-fix 110 is affixed to soft tissue 118 by inserting prongs 116a, 116b. Soft tissue 118 can be, e.g., a muscle, or an internal organ such as an intestinal wall. As they are inserted, prongs 116a, 116b splay, increasing angle α to, e.g., about 30°. Barbs 117a, 117b hold T-fix 110 in place within tissue 118.

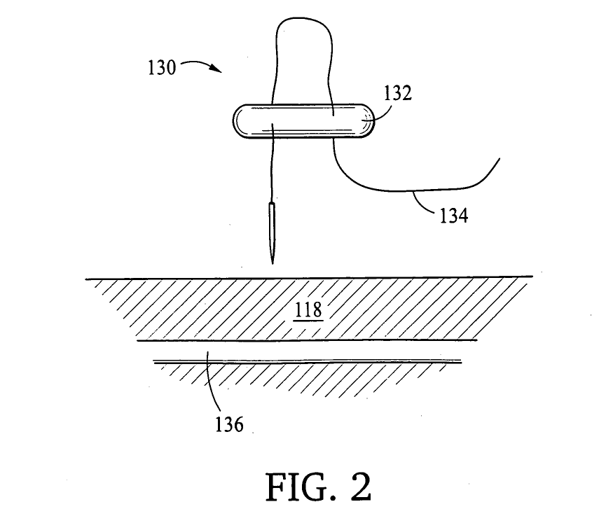

Alternatively, a T-fix can be attached to a desired target site using a suture, rather than a splaying anchor. Referring to FIG. 2, a suture T-fix 130 includes a pellet 132 and a suture 134. T-fix 130 can be attached to a target site by wrapping suture 134 around an internal structure, such as bone 136, or by passing suture 134 through tissue 118.

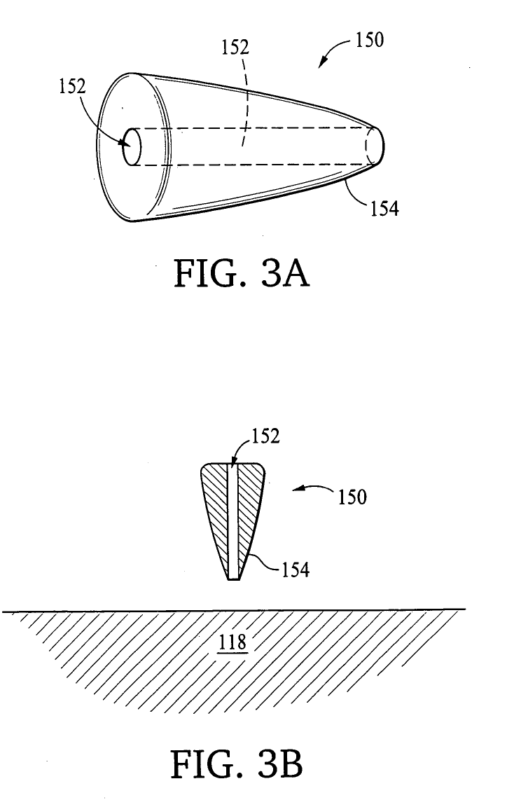

FIGS. 3A and 3B illustrate a plug-shaped implantable device 150. Plug 150 is formed from a drug-polymer mixture, and has a generally conical shape. The plug includes longitudinal through-hole 152 sized and shaped for passage of a guide wire therethrough.

In operation, a guide wire or guide pin is passed into tissue 118 and into contact with, e.g., a bone. The tip of the guide wire makes a small cavity in the bone, and remains pressed against the bone. A drill or other tool is then passed over the guide wire, and used to widen the cavity, such that a dimension of the bone cavity is wide enough to receive, e.g., a portion of distal end 154 of plug 150, or the entire plug 150. After the drill widens the bone cavity, plug 150 is passed over the guide wire and into the cavity. Other known techniques of using guide wires for positioning can also be used.

Guide wires used with plug 150 are generally less than 0.1 inches in diameter, e.g., about 0.031 inches to 0.094 inches, but most frequently about 0.031 to 0.062 inches. Hole 152, therefore, generally has a diameter less than 0.2 inches, e.g., about 0.035 to 0.1 inches.

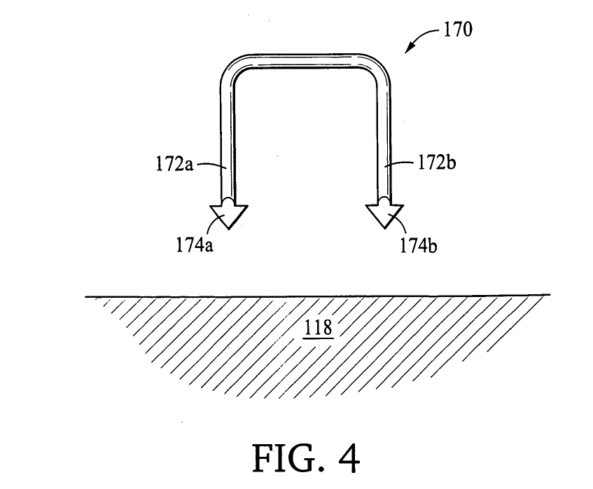

Rather than drilling a cavity in bone, a surgeon can press plug 150 directly into soft tissue, or can wedge the plug into a gap between internal body structures, e.g., between muscle and bone, or between bones in a knee or wrist. Plug 150 can also include a bioabsorbable plastic shell surrounding the drug-polymer mixture to add stability to the plug. Referring to FIG. 4, a staple 170 formed of a drug-polymer mixture has two prongs 172a, 172b. Prongs 172a, 172b have arrow-shaped heads 174a, 174b for engaging soft tissue. As with the T-fix 110 shown in FIG. 1, staple 170 can be attached to various types of internal soft tissue 118, including muscle, and organ walls. Staple 170 can be affixed to soft tissue 118 using a staple gun (not shown) loaded with multiple staples 170.

Staple 170 can be used, e.g., for wound closure after a .surgical procedure. The drug included in the drug-polymer mixture forming the staple can be a pain killer, such as lidocaine, an antibacterial agent to prevent infection, or an agent that promotes healing of the wound.

### Hollow Implantable Devices

The hollow implantable devices generally include a rigid exterior designed to penetrate an internal body structure, such as a bone, muscle, or soft tissue, and a hollow portion or cavity for containing a drug-polymer mixture.

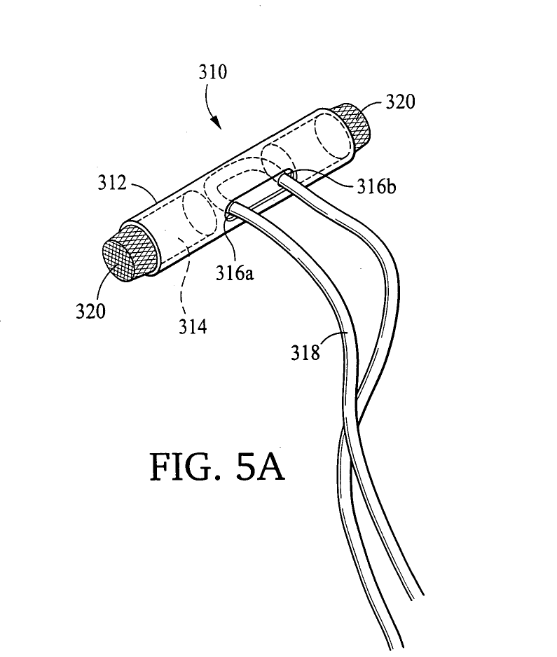

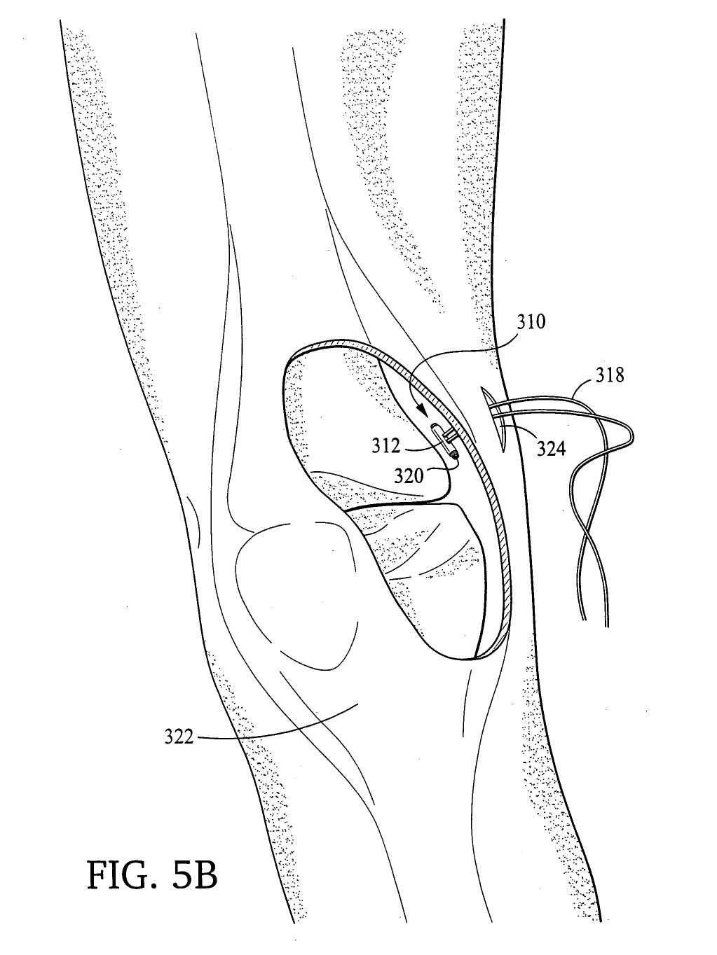

Referring to FIG. 5A, a rigid T-fix 310 includes a cylindrical shell 312 defining a hollow interior 314. Shell 312 also defines two holes 316a, 316b for passage of a suture 318 therethrough. A drug-polymer mixture (not shown), either in powder form or in the form of one or more solid or semi-solid pellets, is loaded into interior 314. A membrane 320 retains the drug-polymer mixture within interior 314 prior to implantation. Membrane 320, however, is permeable to bodily fluids and to the drug, when the drug is dissolved or suspended in bodily fluids.

As shown in FIG. 5B, rigid T-fix 310 is implanted within a location in the body, e.g., a knee 322, by creating a hole 324 in skin and muscle and passing T-fix 310 through hole 324, with the aid of suture 318. Rigid T-fix 310 can then be affixed to soft tissue or tied to a bone, as described above with reference to FIG. 2. Once in place, bodily fluids enter interior 314 through membrane 320, and dissolve the drug-polymer mixture. The drug is then carried out of T-fix 310 by the bodily fluids, and delivered to the nearby target site. Like staple 170, supra, T-fix 310 can be used for wound closure.

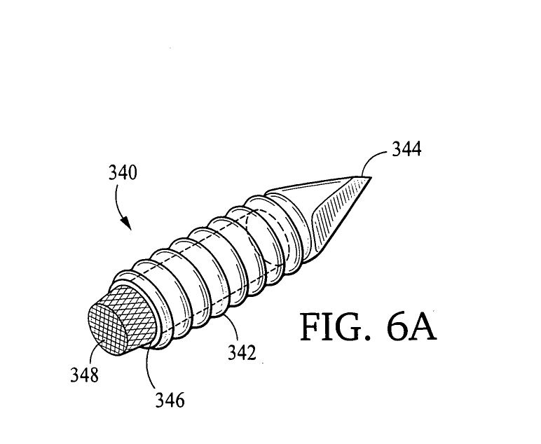

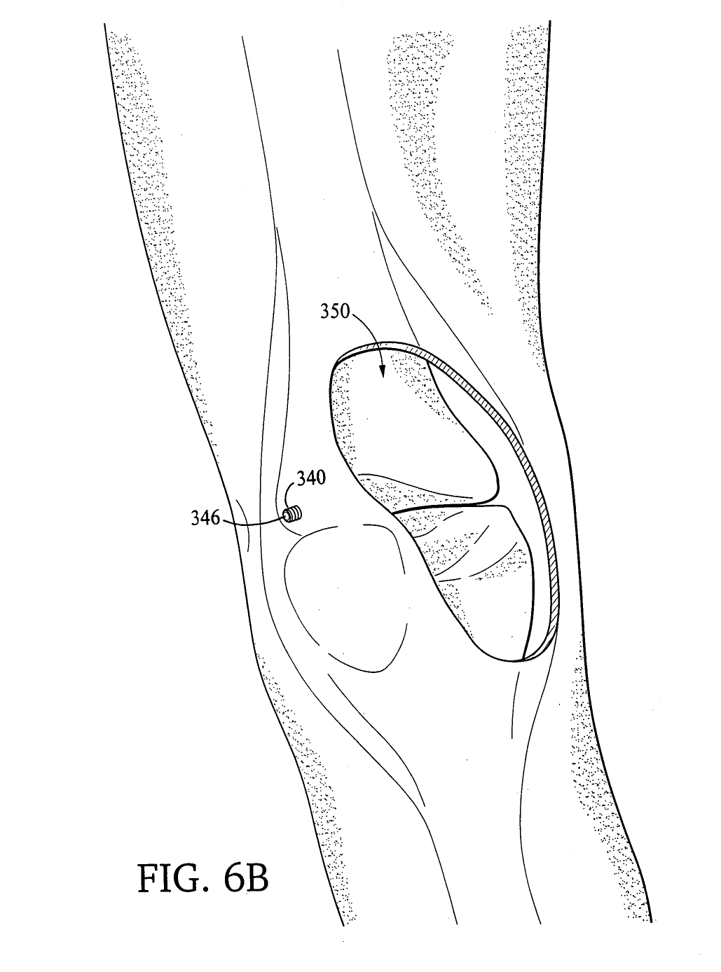

Referring to FIG. 6A, a bone screw 340 includes a threaded shaft 342, a pointed tip 344, and an open end 346. Shaft 342 defines a hollow interior (not shown). As with rigid T-fix 310, a drug-polymer powder or pellet is loaded into the hollow interior, and a membrane 348 covers open end 346 and retains the drug-polymer mixture within the interior. Membrane 348, like membrane 320 is permeable to bodily fluids and to dissolved drug.

Referring to FIG. 6B, bone screw 340 can be drilled into bone, e.g., a knee bone 350, using various drilling tools known in the art. To facilitate implanting bone screw 340 into bone, the opening at end 346 and membrane 348 can be moved to a point along a side 351 of shaft 342. In this arrangement, the hollow interior could be a transverse cavity rather than a longitudinal bore. End 346 could then be solid, and could include a section configured to receive a drilling tool.

Bone screw 340 can also be drilled or manually twisted into soft tissue, such as muscle.

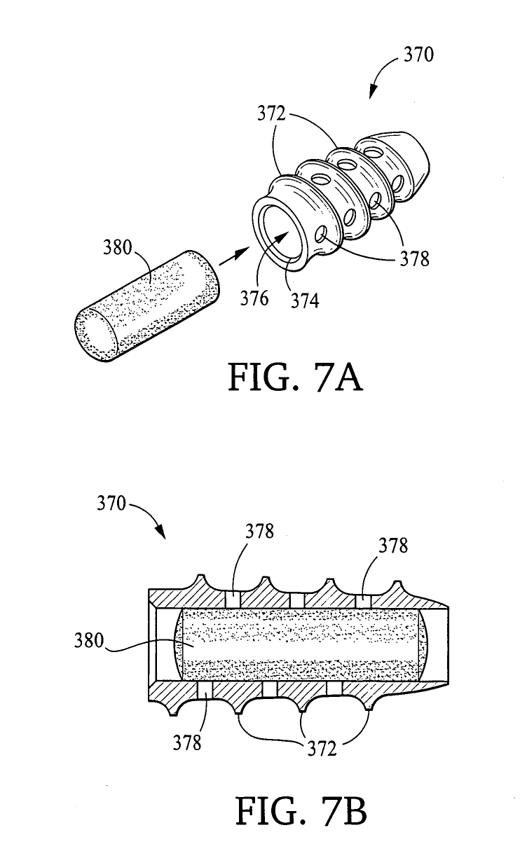

Referring to FIGS. 7A and 7B, a bone screw can also have apertures to release the drug. Bone screw 370 includes threads 372, open end 374, hollow interior 376, and cross holes 378. A pellet 380 made from a drug-polymer mixture is loaded into hollow interior 376 through open end 374. Pellet 380 can be held in place within hollow interior 376 by a membrane, or by sealing open end 374. As shown in FIG. 7B, cross holes 378 expose pellet 380 to the exterior, allowing bodily fluids to reach and dissolve pellet 380.

Bone screws 340 and 370 can be used, e.g., in ligament replacement surgeries, or other surgical procedures that commonly employ bone screws. The drug in the drug-polymer pellets can be an agent that promotes healing, or promotes adhesion of the ligament replacement to bone.

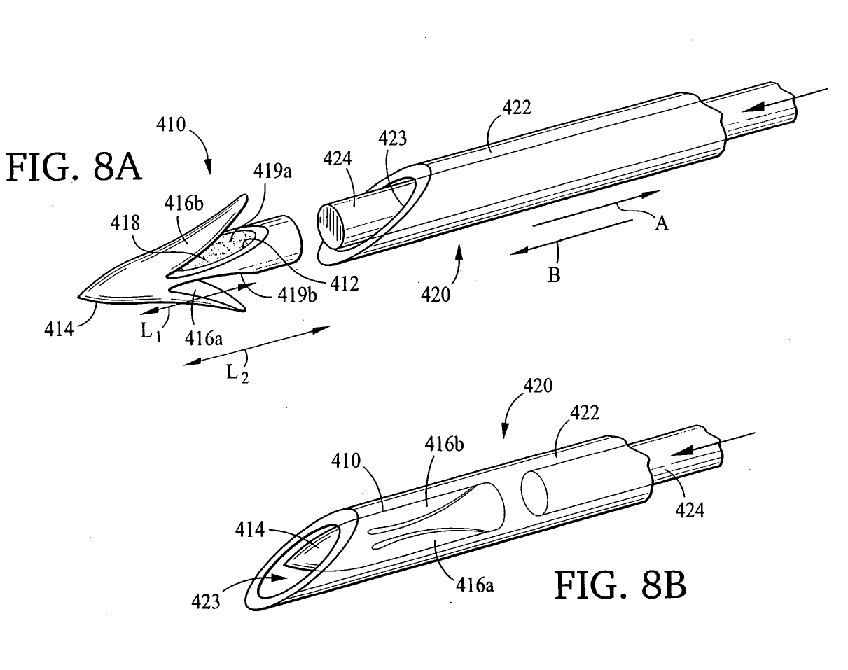

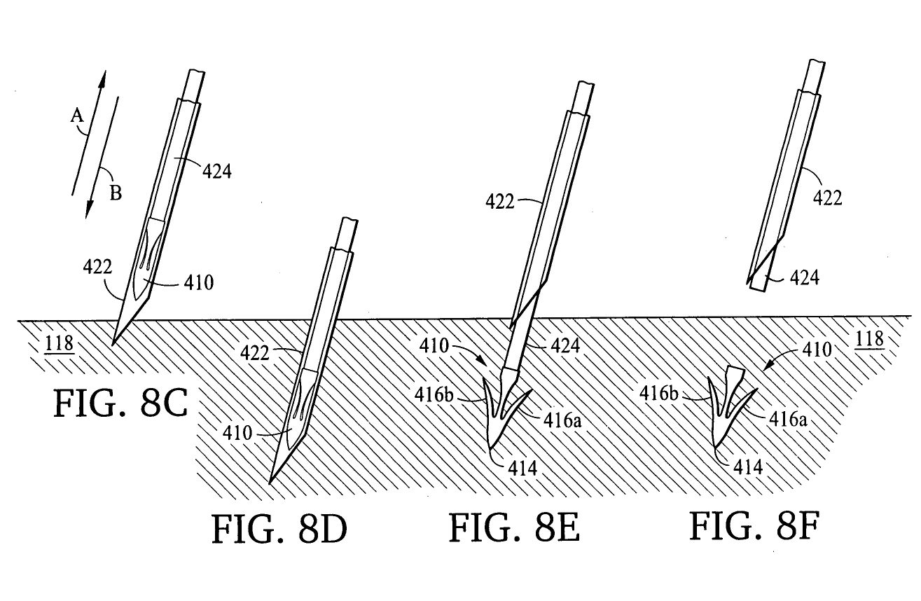

Referring to FIGS. 8A-8F, an implantable plug 410 includes a hollow core 412, a pointed end 414, and retractable engagement wings 416a, 416b. A pellet 418 made from a drug-polymer mixture is loaded into hollow core 412. Pellet 418 has a length L₁ less than the length L₂ of hollow core 412, such that pellet 418 does not entirely fill core 412. Plug 410 includes openings 419a, 419b under wings 416a, 416b which expose pellet 418 to the exterior.

Plug 410 is implanted into soft tissue using a delivery probe 420. Probe 420 has an external shell 422 and hollow interior tube 424. Shell 422 and tube 424 can be made from any rigid material, such as a metal or hard plastic. Interior tube 424 has an external diameter approximately equal to the internal diameter of hollow core 412, such that tube 424 can be snugly fit within core 412. Interior tube 224 is slidable within shell 422 in the direction of arrows A and B. Shell 422 has an open end 423.

In operation, plug 410, with pellet 418 pre-loaded in core 412, is loaded into probe 420 by retracting wings 416a, 416b and inserting core 412 into tube 424. Alternatively, plug 410 can be pre-loaded into probe 420 during manufacture. Tube 424 is then slid in the direction of arrow A to retract plug 410, until plug 410 is fully within shell 422, as shown in FIG. 8C. Next, probe 420 is inserted into soft tissue 118, as shown in FIG. 8D. Tube 424 is then pushed in the direction of arrow B such that plug 410 is pushed out of shell 422, as shown in FIG. 8E. Once plug 410 leaves shell 422, wings 416a, 416b partially expand into tissue 18. Shell 422 and tube 424 are then extracted from tissue 18 by pulling shell 422 and tube 424 in the direction of arrow A, as shown in FIG. 8F. When tube 424 is pulled in the direction of arrow A, wings 416a, 416b engage tissue 18 and prevent plug 410 from moving in the direction of arrow A. Consequently, tube 424 slides out of hollow core 412, leaving plug 410 implanted within tissue 18. Bodily fluids then reach pellet 418 through openings 419a, 419b and slowly dissolve pellet 418, delivering the drug to the nearby target site.

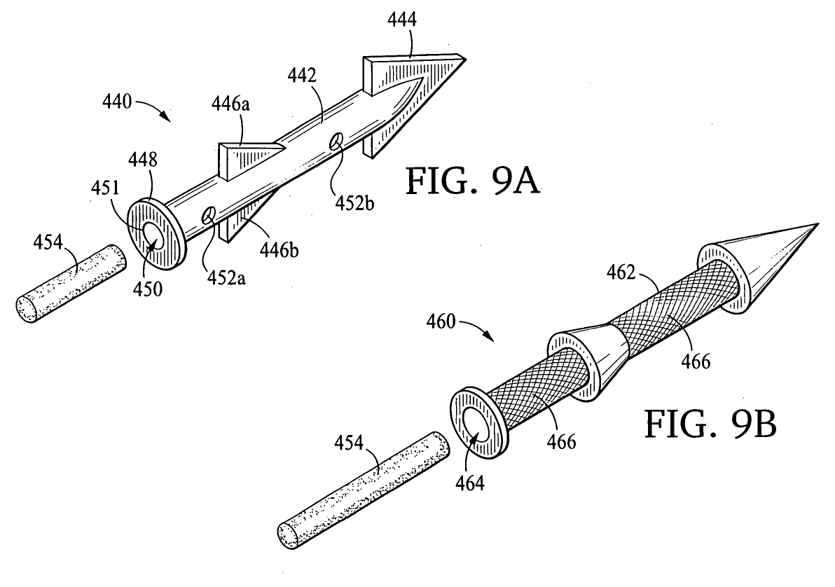

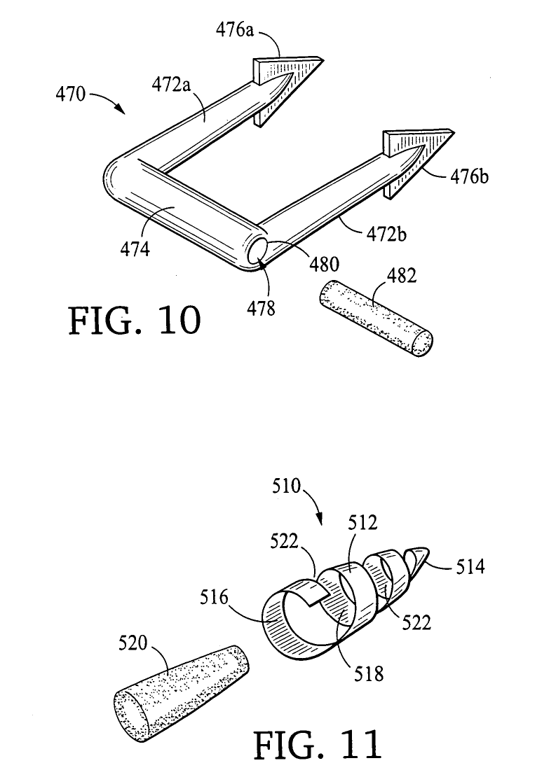

FIG. 9A illustrates a hollowed soft tissue tacker 440. Tacker 440 includes a generally cylindrical body 442, an arrow-shaped head 444, engagement projections 446a, 446b, and an open back end 448. Body 442 defines a hollow, cylindrical cavity 450 communicating with opening 451 of open back end 448. Body 442 also defines four holes, two of which, 452a, 452b, are shown in FIG. 10. The holes allow cavity 450 to communicate with the exterior.

In operation, a pellet 454 made from a drug-polymer mixture is inserted into cavity 450 through back end 448. Pellet 454 can be retained in cavity 450 by covering opening 451 with a permeable membrane (not shown). Tacker 440 is then inserted into soft tissue near a target site, arrow-shaped head 444 first. Head 444 and projections engage the soft tissue, holding tacker 440 in place.

When tacker 440 is inserted into soft tissue, back end 448 remains above the tissue, exposing opening 451 to bodily fluids in a body cavity adjacent to the tissue. Alternatively, tacker 440 can be fully inserted into the tissue. Bodily fluids then enter cavity 450 through opening 451 and through the four holes, dissolving pellet 454 and delivering the drug to the target site.

The soft tissue tacker can also be made from a woven fabric, rather than from an apertured solid shell. Referring to FIG. 9B, a tacker 460 is made from a woven fabric 462, where the threads that form fabric 462 are made from a biodegradable polymer. In tacker 460, bodily fluids enter an internal cavity 464 through gaps 466 in fabric 462, rather than through holes in a solid shell. The tightness of the weave of fabric 462 controls the size of gaps 466 and, therefore, the speed at which the drugs reach the target site. Woven fabrics such as fabric 462 can be used in embodiments other than soft tissue tacker 460 to house drug-polymer pellets.

Referring to FIG. 10, a soft tissue staple 470 includes two penetration arms 472a, 472b, and a connecting arm 474 attaching arm 472a to arm 472b. Penetration arms 472a, 472b include arrow-shaped heads 476a, 476b, and connecting arm 474 defines a cavity 478 and an opening 480. In operation, a pellet 482 made from a drug-polymer mixture is inserted into cavity 478 through opening 480. Pellet 482 can be retained within cavity 478 by covering opening 480 with a permeable membrane (not shown). Once pellet 482 has been inserted, staple 470 is inserted into soft tissue near a target site, arrow-shaped heads 476a, 476b first. When inserted, connecting arm 474 rests against soft tissue, but does not penetrate the tissue. Alternatively, staple 470 can be fully inserted into the tissue. Bodily fluids then enter opening 480 and dissolve pellet 482, delivering the drug to the target site.

FIG. 11 illustrates a helical soft tissue anchor 510. Helical anchor 510 is made from a strip 512 of material, e.g., a polymer, such as polyglycolic acid or polylactic glycolic acid, or a metal, such as stainless steel or titanium, twisted into a helical shape. Helical anchor 510 tapers to a pointed end 514 for penetrating soft tissue. Helical anchor 510 defines an open back 516 and a conical-shaped interior 518 for receiving a tapered pellet 520. In operation, pellet 520 is inserted into interior 518 through open back 516, and helical anchor 510 is then inserted into soft tissue, pointed end 514 first. Helical anchor 510 can be either pushed or twisted into the soft tissue. Bodily fluids then reach pellet 520 through slits 522 and open back 516, dissolving pellet 520 and delivering the drug to a nearby target site.

Alternatively, a helical anchor, e.g., a metal helical anchor, can be machined, and then the drug-polymer mixture can be molded around the helix. In addition, the helical anchor can be manufactured entirely from a drug-polymer mixture that slowly degrades or dissolves to release the drug into bodily fluids over time.

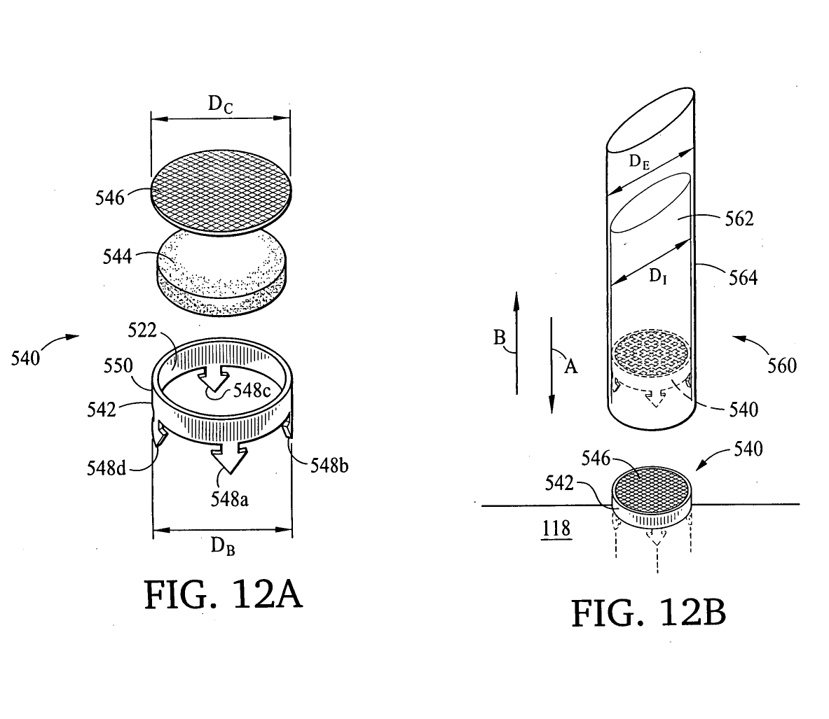

Referring to FIG. 12A, an implantable disk 540 includes a crown-shaped base 542, a wafer 544 made from a drug-polymer mixture, and a permeable membrane cover 546. Cover 546 has a diameter DC approximately equal to a diameter D_{B} of base 542. Base 542 includes four arrow-shaped projections 548a, 548b, 548c, 548d for engaging soft tissue.

In operation, wafer 544 is placed inside rim 550 of base 542. Base 542 can have a shelf (not shown) for receiving wafer 544, or wafer 544 can be attached to the interior 552 of rim 550. Membrane cover 546 is then placed over wafer 544, holding wafer 544 within base 542. A second cover (not shown) can also be placed over wafer 544 on the opposite side of wafer 544. Alternatively, disk 540 can be pre-assembled during manufacture.

After assembly, disk 540 is placed against internal soft tissue by inserting projections 548a, 548b, 548c, 548d into the tissue. Bodily fluids reach wafer 544 through cover 546, dissolve wafer 544, and deliver the drug to a nearby target site.

FIG. 12B illustrates an apparatus 560 for affixing disk 540 to tissue. Apparatus 560 includes an interior cylindrical block 562 slidable within an exterior tube 564. Block 562 and tube 564 can be made from any rigid material, such as a metal or hard plastic. Exterior tube 564 has an inside diameter DE approximately equal to diameter D_{B} of base 542, such that base 542 fits snugly within exterior tube 546. Interior block 562 has a diameter D_{I} less than diameter D_{B}. In operation, disk 540, fully assembled, is loaded into second tube 564. In FIG. 12B, disk 540 is shown in dashed lines inside apparatus 560. Apparatus 560 is then inserted into the body, e.g., through an orifice or a surgically created opening, and pressed against internal soft tissue near a target site. Interior block 562 is then slid in the direction of arrow A, forcing disk 540 out of exterior tube 564 and into the tissue. Apparatus 560 is then withdrawn from the body, leaving disk 540 attached to internal soft tissue.

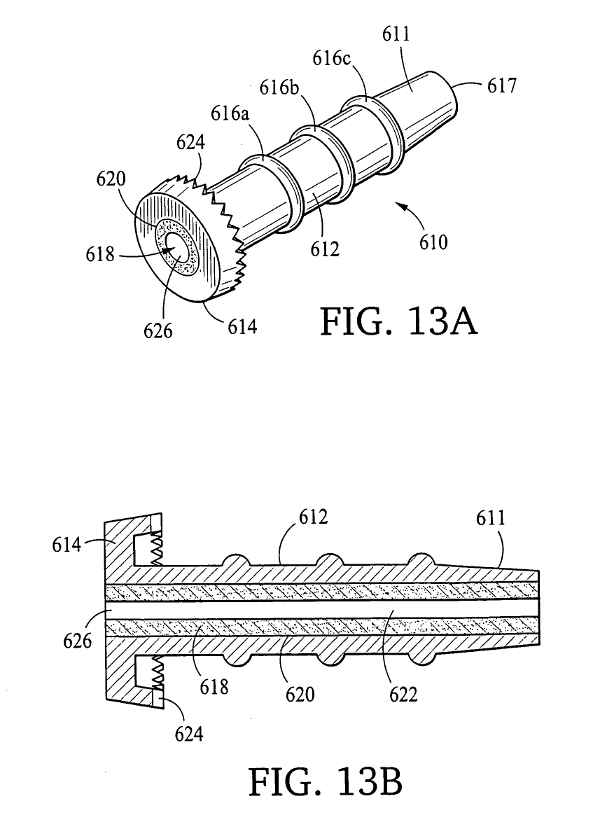

Referring to FIGS. 13A and 13B, a drug delivery tack 610 includes a shell 611 that forms a shaft 612 and a head 614. Shaft 612 includes exterior ribs 616a, 616b, 616c and a tapered end 617. Head 614 includes a jagged edge 624 for engaging soft tissue or bone.

The shell 611 defines a hollow interior bore 618 that extends longitudinally throughout the shaft and the head. A medicament core 620 made from a drug-polymer mixture fills bore 618. A narrow hole 622 is drilled through medicament core 620 for insertion of a guide pin therethrough. Hole 622 has a diameter of, e.g., less than 0.1 inches, and most commonly between about 0.03 and 0.08 inches.

Tack 610 is used to affix soft tissue to a support structure. For example, tack 610 can be used to tension and attach a tendon to muscle, or a ligament to bone. To use tack 610 to attach a ligament to bone, a guide pin (not shown) is inserted through hole 622 until the pin pierces the ligament. The pin is then moved transversely toward the bone, and inserted into a pre-drilled hole in the bone. Tack 610 is then slid over the pin and forced into the hole in the bone, tapered end 617 first, until jagged edge 624 engages the bone (or nearby soft tissue). The guide pin is then removed, leaving the tack in place, and the ligament secured to the bone. A similar procedure is used to attach a tendon to muscle, or other soft tissue to a support structure.

After tack 610 has been inserted and the guide wire has been withdrawn, bodily fluids enter hole 622 through opening 626 and dissolve medicament core 620, delivering the drug to the nearby target site.

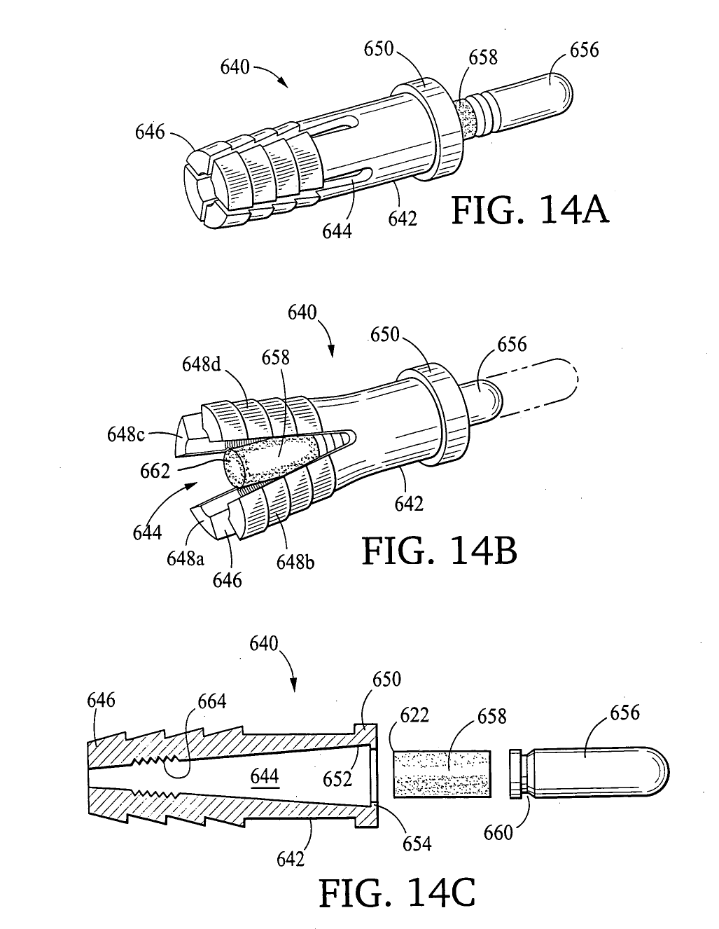

FIGS. 14A-14C illustrate an expansion anchor 640 for delivering a drug. Expansion anchor 640 includes a shaft 642 defining an internal bore 644. Shaft 642 has an end 646 that includes four serrated prongs 648a, 648b, 648c, 648d. Shaft 642 is made from a flexible, bioabsorbable polymer, such as polyglycolic acid or polylactic glycolic acid, allowing radial expansion of bore 644 by, e.g., flexing prongs 648a, 648b, 648c, 648d. Anchor 640 also has a head 650 attached to shaft 642. An interior side 652 of head 650 has a retention ring 654.

A plug 656 holding a drug-polymer pellet 658 is configured to be insertable within bore 644. Plug 656 has a groove 660 sized and shaped to receive retention ring 654.

In operation, plug 656 is first partially inserted into bore 644, until an end 662 of pellet 658 reaches ridges 664 within bore 644. FIG. 14A shows plug 656 partially inserted. Next, anchor 640 is inserted into soft tissue near a target site, until shaft 642 is fully within the tissue. Once anchor 640 has been inserted, plug 656 is pushed further into bore 644, until groove 660 catches ring 654. Pushing plug 656 further into bore 644 causes prongs 648a, 648b, 648c, 648d to flex, radially expanding a portion of bore 644 and exposing pellet 658, as shown in FIG. 14B. Bodily fluids then dissolve pellet 658 and deliver the drug to the nearby target site.

### Materials and Manufacture

The drug-polymer mixture in each of the above implantable devices can be, e.g., a conglomerate of drug-polymer microspheres, a sponge-like polymer matrix in which molecules of drug are embedded, or a solidified drug-polymer mixture, e.g., an emulsion or dispersion.

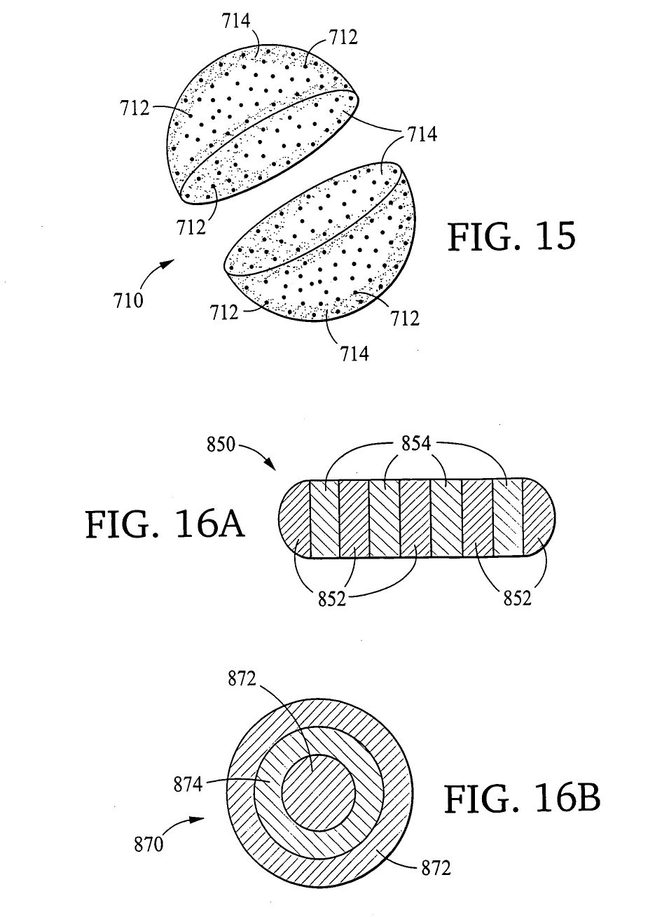

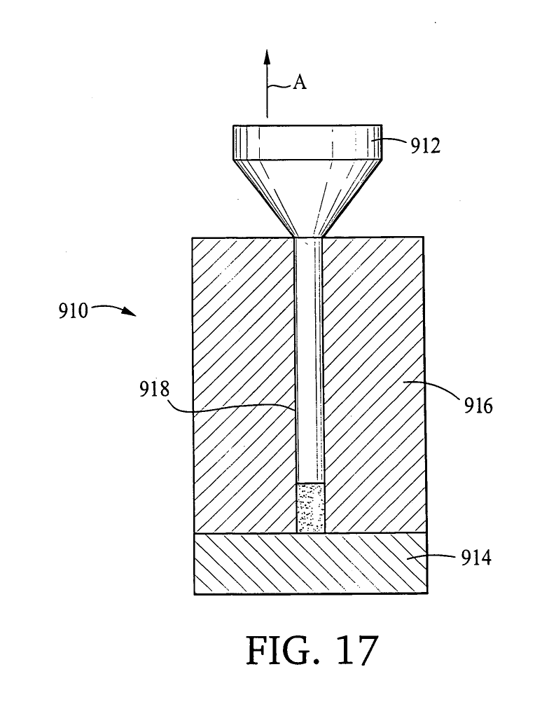

Referring to FIG. 15, in the microsphere conglomerate embodiment, each microsphere 710 includes small amounts of a drug 712 suspended within a polymer substrate 714. The individual microspheres form a “powder” that can be compressed to form the shapes of the shaped implantable devices of FIGS. 1-4, or to form a pellet which can be inserted into the hollow portions of the hollow implantable devices of FIGS. 5-14. Such a conglomerate of drug-polymer microspheres will biodegrade slowly, from the exterior inward, and will therefore steadily release small amounts of the drug over an extended period of time.

The pellet can also be configured to release doses of the drug intermittently. For example, referring to FIG. 15A, in devices where the pellet is only exposed to bodily fluids at one end (e.g., bone screw 340 of FIG. 6), a pellet 850 can be constructed from alternating sectors of drug-polymer mixture 852 and placebo 854. Bodily fluids would dissolve drug-polymer sectors 852 and placebo sectors 854 in succession, causing intermittent release of the drug. Referring to FIG. 15B, pellet 870 is constructed from layers of drug-polymer mixture 872 and placebo 874. Pellet 870 would allow intermittent release of the drug in devices such as T-fix 110 of FIG. 1A, and helical anchor 510 of FIG. 11. In addition, varying layers can be used to release different drugs or different dosages of the same drug.

Alternatively, the microspheres can be left in powder form and loaded into the hollow implantable devices.

A powder including drug-polymer microspheres can be manufactured using known techniques. For example, as described in detail in the Examples below, a drug is dissolved in a polymer-methylene chloride mixture (or a polymer ethyl acetate mixture) to form an inner emulsion. The inner emulsion is then poured into and mixed with an aqueous polyvinyl alcohol solution to form a second emulsion. The resulting double emulsion is then mixed with polyvinyl alcohol and placed on a magnetic stirrer for two-three hours until the methylene chloride evaporates, leaving microspheres. The resulting microspheres are then washed repeatedly using a centrifuge, frozen with liquid nitrogen, and placed in a lyophilizer to form a powder composed of microspheres.

Other known methods of encapsulating drugs within microspheres can also be used. See, e.g., Cohen et al., “Controlled Delivery Systems for Proteins Based on Poly(Lactic/Glycolic Acid) Microspheres,” Pharm. Research, 8:713-20 (1991) (similar to method described above, except that the inner emulsion is poured into and mixed with a polyvinyl alcohol-methylene chloride solution rather than simply a polyvinyl alcohol solution); DeLuca et al., U.S. Pat. Nos. 5,160,745 and 4,741,872 (a vinyl derivative of a polymer, a water soluble monovinyl monomer, and a drug macromolecule are emulsified in water, and the polymer and monomer are co-polymerized such that the macromolecule is entrapped therein); Mathiowitz et al., U.S. Pat. No. 5,718,921 (polymer dissolved in a volatile organic solvent, drug dispersed in the solution, mixture suspended in an organic oil, and the organic solvent extracted, creating microspheres); and Kent et al., U.S. Pat. No. 4,675,189 (polymer water-in-oil solution phase separated by addition of silicone oil, causing polymer to deposit as droplets onto surface of water-polypeptide microdroplets, encapsulating the polypeptide).

In making the drug-polymer microspheres, buffers, such as sucrose and cyclodextrin, can be added. The buffers serve several purposes. First, they act as a cushion for the IL-10 when the microspheres are compressed into pellets, reducing denaturing of the IL-10. Second, the buffers dissolve more quickly than the polymer, creating tunnels in the microspheres to facilitate escape (release) of the IL-10. Inclusion of buffers, therefore, can lead to an initial “burst” of IL-10 release during, e.g., the first 24 hours after implantation, followed by sustained release of a smaller amount of IL-10 over days, weeks, or longer.

Various polymers can be used for encapsulating drugs in microspheres. Preferably, the polymers are biocompatible and degradable when placed within human tissue. Such polymers include, e.g., polyanhydrides, polylactides, polyglycolides, polylactic acid, polyglycolic acid, polyorthoesters, polyorthocarbonates, polyacetals, polymers derived from alpha hydroxycarboxylic acids and lactones, polymers derived from condensation of divinyl ethers and polyols, e-caprolactone polymers, and various other polymers described in the above incorporated references. In addition, co-polymers of some of the above polymers, such as poly(DL-lactide-co-glycolide) can be used to encapsulate certain drugs.

Various drugs and combinations of drugs can be encapsulated by polymers for delivery using the claimed devices. For example, anti-inflammatory agents, such as down-regulatory cytokines, can be used to treat inflammatory disease, as described in the Examples below. Pain medications, such as lidocaine, can be used to treat localized pain. Other possible drugs include platelet derived growth factor, antibiotics, hormones, prostaglandins, insulin, adrenalin, xylocaine, morphine, corticoid compounds, atropine, cytostatic compounds, estrogen, androgen, interleukins, digitoxin, biotin, testosterone, heparin, cyclosporin, penicillin, vitamins, anti-platelet activating agents, somatostatin, SOMATRIPTAN™, triptorelin, diazepam, other protein based drugs, peptide sequences (which are generally more heat resistant and last longer than full proteins), nucleic acid based drugs and therapies, and other drugs described in the incorporated references.

Instead of encapsulating the drug within polymeric microspheres, the polymer and drug can simply be mixed together in powdered form, and then compressed into pellets. Non-microsphere pellets would also release small amounts of the drug steadily, over an extended period of time, as the polymer in the pellet biodegrades. Alternatively, liquid or semi-solid drugs and polymers can be mixed and then extruded into rods that can be cut into short pellets.

To create a non-microsphere drug-polymer mixture, an emulsion including a drug and a polymer can be frozen with liquid nitrogen and then placed in a lyophilizer. This process is similar to the microsphere formation process described in detail in the Examples below, except that the drug polymer emulsion is not stirred with a magnetic stirrer.

Alternatively, a drug can be dissolved in a mixture of methylene chloride and ethylene vinyl acetate copolymer. A small amount of the resulting solution is then placed in a mold that has been frozen with liquid nitrogen. The frozen mold is then placed in a vacuum chamber to dissolve the solvent, leaving only a film of the ethylene vinyl acetate and the drug. The film, which is typically rubbery and somewhat adhesive, can be rolled tightly into a pellet for insertion into an implantable device.

Other techniques for mixing drugs and polymers into sustained release formulations can also be used. See, e.g., Cohen et al., “Sintering Techniques for the Preparation of Polymer Matrices for the Controlled Release of Macromolecules,” J. Pharm. Sciences, 73:1034-37 (1984) Briefly, drug and polymer powders are mixed at a temperature below the glass transition point of the polymer. The resulting mixture is then compressed at a temperature above the glass transition point, forming the matrix.

In the non-microsphere embodiment, many of the polymers mentioned above can be used, in addition to other polymers, such as ethylene-vinyl acetate copolymer and some non-biodegradable polymers.

The microspheres or the non-microsphere drug-polymer mixture are compressed into shapes or pellets using simple molds and a press, e.g., a Carver press. The amount of pressure required to shape a powder into an implantable device having a desired shape will depend on the size of the device and the particular drug-polymer mixture.

The rigid exteriors of the devices illustrated in FIGS. 5-14 can be made from a variety of materials, depending on the nature of the implantable device. The rigid exteriors of the bone screws of FIGS. 6 and 7, for example, are typically made from a biocompatible metal, such as titanium, cobalt, chromium, stainless steel, or other alloys. The rigid exteriors of the devices of FIGS. 5 and 8-14, however, can be manufactured from a rigid, biodegradable polymer, such as polyglycolic acid or polylactic glycolic acid, a hard, non-binding surgical grade plastic,such as DELRIN™, or a non-biodegradable polymer, ceramic, or metal.

The shaped implantable devices of FIGS. 1-4 can be formed by compressing drug-polymer powders into the desired shape, as described below with reference to FIG. 16. The hollowed implantable devices of FIGS. 5-14 can be formed using techniques known in the art, including deposition of a molten polymer into a mold, or extrusion. The devices can also be formed from several separate pieces melded together using heat.

The permeable membranes of the embodiments of, e.g., FIGS. 5, 6, and 12 can made from, e.g., any membrane material known in the art. The size and density of the pores in the membranes can be varied, depending on the drug and the desired drug delivery rate. In general, the membranes will have micron ratings of greater than 0.5 (for filtering suspended solids, but not dissolved large molecules). Other micron sizes are possible, depending on the application. Membranes can be purchased from, e.g., RGF ENVIRONMENTAL, West Palm Beach, Fla.

The sizes of the devices of FIGS. 1-14 can vary. Generally, the longest dimension of each device will range from about 1.5 mm to 1 cm or larger, e.g., 2 mm, 5 mm, 1 cm, 2 cm, or 5 cm.

The invention is further described in the following Examples, which do not limit the scope of the invention described in the claims.

### EXAMPLES

In the following Examples, interleukin-10 (“IL-10”) was encapsulated in microspheres of 50:50 poly(DL-lactide-co-glycolide). The resulting microsphere powder was compressed into pellets, and also tested for biological activity. The results of these Examples establish that IL-10 can be incorporated into implantable devices such as those described above for localized, controlled release of IL-10 directly to a site of inflammation.

### EXAMPLE 1

### Encapsulation of IL-10 within Polymer Microspheres

In three separate experiments, IL-10 was encapsulated within 50:50 poly(DL-lactide-co-glycolide) microspheres.

In each experiment, the materials and equipment were as described in Table 1.

TABLE 1

| MATERIAL/DEVICE | DESCRIPTION |
|---|---|
| Polymer powder (50:50 | BOEHRINGER INGELHEIM (Henley |
| poly(DL-lactide-co- | Chemicals), Cat. No. RG503. |
| glycolide)) | |
| Polyvinyl alcohol | ALDRICH CHEMICAL CO., Cat. |
| | No. 18,953-7; 96% hydrolized |
| Ethyl Acetate | ALDRICH CHEMICAL CO., Cat. |
| | No. 27,098-9; 99.8% |
| | anhydrous |
| Recombinant Human IL-10 | ENDOGEN, INC., Cat. No. R- |
| | IL10-25 |
| Methylene Chloride | ALDRICH, Cat. No. 27,099-7; |
| (Dichloromethane) | 99.8% anhydrous |
| Human Serum Albumin | CALBIOCHEM, Cat. NO. 12666; |
| | Type: Fraction V |
| Hydroxypropyl Beta | AM. MAIZE-PRODUCTS CO., |
| CycloDextrin | Hammond, Indiana |
| Sonicator | VibraCell ™ Sonicator |
| Homogenizer | Silverson L4R Homogenizer |
| Centrifuge | IEC model Centra GP8 |
| Lyophilizing chamber | Labconco |
| IL-10 (recombinant human | ENDOGEN, Cat. No. R-IL10-25 |
| interleukin 10) | |
| Methylene Chloride | ALDRICH, Cat. No. 27, 099-7; |
| (Dichloromethane) | 99.8% anhydrous |
| HSA (human serum albumin) | CALBIOCHEM, Cat. No. 12666; |
| | Type: Fraction V |

The remaining chemicals and equipment are standard laboratory supplies available from numerous sources.

### First Experiment

First, two separate sets of IL-10 solution, polymer powder solution, and homogenized polyvinyl alcohol solution were prepared as follows.

Ten micrograms of IL-10 previously bulked with bovine serum albumin (“BSA”), and 25 micrograms of pure IL-10 were placed in separate vials. Next, 1 ml of chilled phosphate buffered solution, pH 7.4 (“PBS”), was added to the 25 microgram vial, and 400 μl of PBS was added to the 10 microgram vial. The solutions were mixed and then chilled.

Two 50 mg of samples of 50:50 poly(DL-lactide-co-glycolide) (“polymer powder”) were placed in two separate test tubes. One ml of methylene chloride was added to each tube, and the resulting polymer solutions were chilled.

Two separate beakers, one labeled “w/BSA” and one labeled “w/o BSA” were prepared. To each beaker, 100 ml of 1% polyvinyl alcohol was added and placed in a homogenizer at 5800 rpm for several minutes.

Next, 100 μl of the 10 microgram IL-10 solution was added to the first polymer solution test tube. The test tube was sonic pulsed in the sonicator for about 5 pulses (40% duty cycle), and the resulting emulsion was added to the beaker labeled “w/BSA” while still homogenizing at 5800 rpm. Homogenization was continued for an additional 1 minute, and the beaker was then moved to a magnetic stirrer set at a speed of about 4.5. Similarly, 100 microlitres of the 25 microgram IL-10 solution was added to the second polymer solution test tube, pulsed, and added to the beaker labeled “w/o BSA.” This beaker was also homogenized for an additional 1 minute, and then moved to the magnetic stirrer. At this point, microspheres could already be observed through a microscope.

After two hours in the stirrer, the two beakers were removed from the magnetic stirrer. The resulting solutions were then poured into four 50 ml centrifuge vials: two labeled “w/BSA” and two labeled “w/o BSA.” The vials were centrifuged at 1500 rpm (program 6). After centrifuging for 5 minutes, the vials were removed, the liquid was poured off the top, and distilled water was added to return the total volume in each vial to about 30 ml. The vials were then centrifuged for an additional five minutes. Once again, the vials were removed, the liquid was poured from the top, and distilled water was added to return the volume to 20 ml. The vials were centrifuged again for 5 minutes, and distilled water was added to bring the total volume to 5-10 ml per vial.

The vials were then dipped into a bucket of liquid nitrogen until frozen, covered with KIMWIPES and a rubber band, and placed in a lyophilizing chamber. The chamber was attached to the lyophilizer, and the vents to vacuum were opened until the reading reached 100 microns Hg.

The result was 70 mg of fine, white powder composed of microspheres of 50:50 poly(DL-lactide-co-glycolide) entrapping IL-10 (40 mg of microspheres with BSA, 30 mg of microspheres without BSA).

### Second Experiment

In this experiment, sucrose and CycloDextrin buffers were added to polymer/IL-10 microsphere mixtures. The sugar buffers serve two purposes. First, they act as a cushion during pressing of IL-10 powder into pellets, thereby protecting the IL-10 from being denatured by the pressure. Second, the sugar buffers, which are larger than IL-10 molecules, form “tunnels” in the microsphere pellets after the powder is compressed, facilitating release of the IL-10 after implantation.

The experiment was performed as follows. First, 100 ml of 1% polyvinyl alcohol was poured into six beakers and chilled using an ice bath. The beakers were labeled “MeCl/std,” “MeCl/su,” “MeCl/CD,” “EtAc/std,” “EtAc/su,” and “EtAc/CD.”

Ten grams of powdered human serum albumin (“HSA”) was combined with distilled water to make a stock HSA solution having a concentration of 10 mg/1 ml. (The HSA helps protect the IL-10 from becoming denatured.) One hundred μl of the stock HSA was combined with 400 μl of distilled water and added to the vial containing the 25 μg of IL-10. The mixture was mixed gently using a VORTEX GENIE, and then chilled.

Six 50 mg samples of polymer powder were placed in six test tubes labeled “MeCl/std”, “MeCl/su”, “MeCl/CD”, “EtAc/std”, “EtAc/su”, and “EtAc/CD.” One ml of methylene chloride was then added to each of the three “MeCl” test tubes, and 1 ml of ethyl acetate was added to each of the three “EtAc” test tubes. The tubes were then chilled. The methylene chloride easily dissolved the polymer, but the ethyl acetate did not. The VORTEX GENIE was used to help dissolve the polymer in each test tube.

Next, 100 μl of distilled water was poured into three vials labeled “std,” “su,” and “CD.” Ten mg of sucrose was added to the “su” vial and 10 mg of CycloDextrin was added to the “CD” vial. The contents of the vials were then mixed. Approximately 125 μl of the IL-10 solution were added into each of the 3 vials. The contents were mixed gently using the Vortex Genie only on low settings.

The six beakers (each containing 100 ml PVA) were placed in the homogenizer at 4600-4700 rpm for several minutes.

Approximately half of the “std” IL-10 solution, about 112 μl, was added to the “MeCl/std” polymer solution test tube, and the other half was added to the “EtAc/std” polymer solution test tube. Similarly, the “su” and “CD” IL-10 solutions were divided into the corresponding polymer solutions: “MeCl/su” and “EtAc/su,” and “MeCl/CD” and “EtAc/CD.” The test tubes were then sonic pulsed in the sonicator (20% duty cycle) for 4 pulses while keeping them on ice.

Each emulsion was added to the correspondingly labeled PVA-filled beaker while still homogenizing at 4600-4700 rpm. Homogenization was continued for an additional 1 minute. The beakers were then moved to a magnetic stirrer, set at a speed of 6. At this point microspheres could already be observed through a microscope.

After 2-3 hours, the beakers were removed from the magnetic stirrer. Twelve 50 ml centrifuge vials were labeled as follows: “MeCl/std 1,” “MeCl/std 2,” “MeCl/su 1,” “MeCl/su 2,” “MeCl/CD 1,” “MeCl/CD 2,” “EtAc/std 1,” “EtAc/std 2,” “EtAc/su 1,” “EtAc/su 2,” “EtAc/CD 1,” and “EtAc/CD 2.” The solutions from the beakers were poured into the corresponding 12 vials. The vials were then centrifuged for 5 minutes at 1500 rpm (program 6).

Next, the vials were removed from the centrifuge and liquid was carefully poured off the top. The solid (microspheres) residue of the two vials marked “MeCl/std 1” and “MeCl/std 2” were combined by adding some distilled water to the vials to re-suspend the microspheres, and then pouring one vial into the other. In the consolidated vial, more distilled water was added to reach a total volume of about 30 ml. This “washing” process was repeated for the other 10 vials. After all were combined, only 6 vials remained. The vials were then centrifuged as before. The microspheres were then washed again (liquid poured off the top and spheres re-suspended with distilled water), and additional distilled water was added to reach a total volume of about 25 ml in each of the six vials. The vials were then centrifuged and washed once more, and distilled water was added to reach a total volume of about 5 ml in each of the 6 vials.

The vials were then dipped into a bucket of liquid nitrogen until frozen, covered with KIMWIPES and a rubber band, and placed in a lyophilizing chamber. The chamber was attached to the lyophilizer, and the vents to vacuum were opened until the reading reached 100 microns Hg.

The remaining IL-10 solution was saved for the biological activity tests described below.

### Third Experiment

In this experiment, two different variations of polymer/IL-10 microsphere mixtures were created.

First, 100 ml of 1% polyvinyl alcohol was poured into four beakers and chilled using an ice bath. The beakers were labeled “MeCl/su A,” “MeCl/su B,” “MeCl/CD A,” “MeCl/CD B.”

Next, as in the second experiment, 10 grams of powdered HSA was combined with distilled water to make a stock HSA solution having a concentration of 10 mg/1 ml. One hundred μl of the stock HSA was combined with 400 μl of distilled water and added to the vial containing the 25 μg of IL-10. The mixture was mixed gently using a Vortex Genie, and then chilled.

Four 100 mg samples of polymer powder were placed in four test tubes labeled “MeCl/su A,” “MeCl/su,B,” “MeCl/CD A,” “MeCl/CD B.” Two ml of methylene chloride were then added to each of the two MeCl test tubes. The Vortex Genie was used to help dissolve the polymer.

Next, 20 mg of sucrose was added to the “su” vial, 20 mg of CycloDextrin was added to the “CD” vial, and about 250 μl of the IL-10 solution was added to each of the two vials. The contents were then mixed gently using the Vortex Genie on low settings only. About 250 μl of distilled water was added to each vial.

The four beakers (each containing 100 ml PVA) were placed in homogenizer at 4600-4700 rpm, for several minutes.

Approximately half of the “su” IL-10 solution, about 250 μl, was added to the “MeCl/su A” polymer solution test tube, and the other half was added to the “MeCl/su B” polymer solution test tube. Similarly, the “CD” IL-10 solution was divided into the corresponding polymer solutions: “MeCl/CD A” and “MeCl/CD B.” The test tubes were then sonic pulsed in the sonicator (20% duty cycle) for 5-6 pulses while keeping them on ice.

Each emulsion was added to the correspondingly labeled PVA-filled beaker while still homogenizing at 4600-4700 rpm. Homogenization was continued for an additional 1 minute. The beakers were then moved to a magnetic stirrer, set at a speed of 6. At this point microspheres could already be observed through a microscope.

After 2-3 hours, the beakers were removed from the magnetic stirrer. Eight 50 ml centrifuge vials were labeled as follows: “MeCl/su A1,” “MeCl/su A2,” “MeCl/su B1,” “MeCl/su B2,” “MeCl/CD A1,” “MeCl/CD A2,” “MeCl/CD B1,” and “MeCl/CD B2.” The solutions from the beakers were poured into the corresponding 8 vials. The vials were then centrifuged for 5 minutes at 1500 rpm (program 6).

Next, the vials were removed from the centrifuge and liquid was carefully poured off the top. The solid residue (microspheres) of the two vials marked “MeCl/su A1” and “MeCl/su A2” were combined by adding some distilled water to the vials to re-suspend the microspheres, and then pouring one vial into the other. In the consolidated vial, more distilled water was added to reach a total volume of about 30 ml. This “washing” process was repeated for the other 6 vials. After all were combined, only 4 vials remained. The vials were then centrifuged as before. The microspheres were then washed again (liquid poured off the top and spheres re-suspended with distilled water), and additional distilled water was added to reach a total volume of about 25 ml in each of the four vials. The vials were then centrifuged and washed once more, and distilled water was added to reach a total volume of about 5 ml in each of the 4 vials.

The vials were then dipped into a bucket of liquid nitrogen until frozen, covered with KIMWIPES and a rubber band, and placed in a lyophilizing chamber. The chamber was attached to the lyophilizer, and the vents to vacuum were opened until the reading reached 100 microns Hg.

### EXAMPLE 2

### Compression of IL-10/Polymer Powder into Pellets

Microsphere powder obtained from the first experiment of Example 1 was compressed into four disk-shaped pellets as follows. Referring to FIG. 16, a disk-shaped mold 910 includes a removable top 912, a removable bottom 914, and a body 916 defining a bore 918. To load the mold, top 912 was removed from bore 918 in the direction of arrow A, and 10 mg of microsphere powder containing BSA was loaded into bore 918. Top 912 was reinserted into bore 918 over the powder, and twisted to compress the powder. Mold 910 was then subjected to 1500 pounds of force from a Carver Press (not shown) for seven minutes, creating a 5 mm diameter flat disk pellet.

Five additional pellets were prepared in the same manner: one more using microsphere powder with BSA, two using microsphere powder without BSA, and two using pure polymer powder. Mold 910 was cleaned between each use with methylene chloride.

To test the structural integrity of the pellets, sutures were successfully passed through each of the four different microsphere pellets.

### EXAMPLE 3

### Testing of Polymer/IL-10 Microspheres for Biological Activity

A series of tests were performed to verify that microspheres formed by the second and third experiments of Example 1 released encapsulated IL-10 when placed in a biological environment, and that the released IL-10 will inhibit production of TNF-α.

To test release of IL-10 in a biological medium, IL-10 microspheres were first incubated with Dulbecco's Modified Eagle Medium (DMEM) at 37° C. The medium and microspheres were kept on a rocker to prevent the microspheres from settling. After a predetermined amount of time (e.g., 3 hours) the medium and microspheres were removed and centrifuged. The supernatant was collected, and an enzyme-linked immunosorbent assay (ELISA) was used to measure the amount of IL-10 in the supernatant. The amount of IL-10 found in the supernatant was recorded as IL-10 released during the “0-3 hrs” interval. The microspheres were then returned to the medium and incubated further. At another predetermined time (e.g., after 21 additional hours), the medium and microspheres were removed and centrifuged again, and the amount of IL-10 found in the supernatant was recorded as IL-10 released during the “3-24 hrs” interval. The process was repeated to measure IL-10 released;during subsequent intervals. The data tables for each release experiment, therefore, show how much IL-10 was released by each type of microsphere, and when the IL-10 was released.

ELISAs were also used to measure degradation of IL-10 in the DMEM over the various time intervals. For the degradation experiments, loose IL-10 (not encapsulated in microspheres or mixed with polymer) was placed in the DMEM at an initial concentration of, e.g., 200 ng/ml. At the end of each time interval, the concentration of IL-10 remaining was measured by removing and testing a small sample of the medium.

To test biological activity of the released IL-10, IL-10 was taken from the supernatants used to perform the ELISA tests, and added to monocytes to achieve a final concentration of 1 ng/ml. Some IL-10 bound to the IL-10 receptors of the monocytes and became incorporated into the cells. The final cell concentration was 1×10⁶ cells/ml of DMEM.

After 1.5-2 hours, the monocytes were first stimulated with a concentration of 100 Units/ml of interferon gamma (IFN-γ) and then with a concentration of 20 μg/ml of muramyl dipeptide (MDP). The IFN-γ increases MDP receptor expression so that the MDP can bind readily with the cells. Once the MDP binds to the cell and is incorporated, it attempts to turn on the TNF-α gene. However, if active IL-10 is already in the cell, it will block the TNF-α gene from turning on and producing TNF-α.

After 16 hours, the cells were harvested and the culture supernatant was collected. The TNF-α levels of the cells and supernatant were then tested by a cloned mouse fibrosarcoma cell line (LM) bioassay, to determine the extent to which TNF-α production was inhibited.

### Results for Testing of Microspheres Formed in Second Experiment of Example 1

The following ELISA data (Table 2) were obtained for microspheres formed in the second powder formation experiment of Example 1. The beginning concentration of It-10 (in the DMEM) was about 200 ng/ml, for both the microsphere encapsulated IL-10 and the loose IL-10.

TABLE 2

| | |
|---|---|
| | IL-10 released (ng/ml) per |
| | amount of time in culture |
| ELISA Results | medium (DMEM) at 37° C. |

| Sample # | Name | 0-24 hrs. | 24-48 hrs. |
|---|---|---|---|
| 1 | MeCl/std | 35 | 0.024 |
| 2 | MeCl/su | 113 | 0.8 |
| 3 | MeCl/CD | 101 | 0.4 |
| 4 | EtAc/std | 15 | 0.3 |
| 5 | EtAc/su | 61 | 1.3 |
| 6 | EtAc/CD | 65 | 1.1 |
| 7 | Loose IL-10 | 290 | 87 |

These results show that the MeCl/su and MeCl/CD microspheres release more IL-10 than the other samples during first 24 hours, but the EtAc/su and EtAc/CD microspheres release the most IL-10 over a two day period. The concentration of the loose IL-10 actually increased during the first 24, hours due to evaporation of DMEM. The concentration dropped during the second 24 hours, however, due to degradation of IL-10.

The LM bioassay results for the IL-10 released during the 0-24 hrs interval are shown in Table 3 below.

TABLE 3

| | | | TNF-α | |
|---|---|---|---|---|
| | | TNF-α | Membrane | TNF-α |
| | | Supernate | Bound | Total |
| Sample # | Name | (pg/ml) | (pg/ml) | (pg/ml) |
| 1 | Control | 47,199 | 906 | 48,105 |
| 2 | MeCl/std | 29,207 | 576 | 29,783 |
| 3 | MeCl/su | 31,439 | 0 | 31,439 |
| 4 | MeCl/CD | 19,999 | 0 | 19,999 |
| 5 | EtAc/std | 29,776 | 0 | 29,776 |
| 6 | EtAc/su | 32,736 | 616 | 33,352 |
| 7 | EtAc/CD | 21,786 | 0 | 21,786 |
| 8 | IL-10 | 22,951 | 0 | 22,951 |
| | Control | | | |

Sample 1, the control, shows that 48,105 pg/ml of TNF-α are produced by monocytes stimulated with MDP and IFN-γ. To set a benchmark for the effectiveness of IL-10 on reducing TNF-α production, pure IL-10 was added to Sample 8, the IL-10 Control. This benchmark shows that the IL-10 reduces the TNF-α level from 48,105 to 22,951 pg/ml. Samples 2-7 represent the various microspheres. All lowered the TNF-α levels. The best results were from the MeCl/CD and EtAc/CD microspheres which actually lowered the TNF-α levels below the benchmark.

The amount of IL-10 collected from each sample after 48 hours was not enough to run a bioassay. The second experiment of Example 1, therefore, was repeated. In this second run, IL-10 release data was gathered for the 0-3 hours interval, the 3-24 hours interval, the 1-5 days interval, and the 5-12 days interval. The ELISA results for this second run are shown in Table 4 below:

TABLE 4

| | | IL-10 released (ng/ml) per amount | | | |
|---|---|---|---|---|---|
| | | of time in culture medium (DMEM) at | | | |
| | | 37° C. | | | |
| ELISA Results | | 0-3 | 3-24 | 1-5 | 5-12 |
| Sample # | Name | hrs. | hrs. | days | days |
| 1 | MeCl/std | 30 | 0.65 | 0.076 | 0.009 |
| 2 | MeCl/su | 82 | 13 | 1.26 | 0.178 |
| 3 | MeCl/CD | 90 | 5.6 | 0.39 | 0.066 |
| 4 | EtAc/std | 18 | 1.4 | 0.084 | 0.023 |
| 5 | EtAc/su | 74 | 5.5 | 0.466 | 0.095 |
| 6 | EtAc/CD | 77 | 8 | 1.03 | 0.135 |

These results show sustained release of the IL-10 even though a large portion of the IL-10 is released in the first three hours. Most likely, the initial burst of IL-10 release is caused by the inclusion of HSA and the CD and SU sugar buffers. The buffers are fairly large molecules, and they tend to dissolve faster than the polymer. As the buffers dissolve, they create tunnels in the microspheres, causing an initial burst of IL-10 release from the IL-10 mixed with the buffers. Once the buffers have dissolved, the IL-10 mixed with the polymer escapes at a steady rate.

The LM bioassay results for the IL-10 released during the 0-3 hours and the 3-24 hours intervals of the second run are shown in Table 5 below.

TABLE 5

| | | % Inhibition | % Inhibition |
|---|---|---|---|
| Sample # | Name | 0-3 hrs. | 3-24 hrs. |
| 1 | MeCl/std | 6 | |
| 2 | MeCl/su | 40 | 19 |
| 3 | MeCl/CD | 10 | 17 |
| 4 | EtAc/std | 0 | |
| 5 | EtAc/su | 20 | 14 |
| 6 | EtAc/CD | 28 | 19 |
| 7 | IL-10 | 82 | 60 |
| | Control | | |

In this test, the MeCl/su and EtAc/CD microspheres were most effective, especially during the first 3 hours.

In this second testing run, bioactivity was much lower than in the first run, so another testing experiment was performed. This third run began with an IL-10 concentration of 500 ng/ml. The ELISA results are shown below in Table 6:

TABLE 6

| | | IL-10 released (ng/ml) per |
|---|---|---|
| | | amount of time in culture |
| ELISA Results | | medium (DMEM) at 37° C. |
| Sample # | Name | 0-24 hrs. |
| 1 | MeCl/su | 340 |
| 2 | MeCl/CD | 132 |
| 3 | EtAc/su | 103 |
| 4 | EtAc/CD | 102 |
| 5 | Loose IL-10 | 905 |

These results again show that the MeCl microspheres release more IL-10 during the first 24 hours than the EtAc microspheres. As before, the concentration of the loose IL-10 increased due to evaporation of DMEM.

In this third run, appropriate amounts of IL-10 were taken from the same supernatant used to perform the ELISAs and added to monocytes to achieve a final concentration of 10 ng/ml, rather than 1 ng/ml. The LM bioassay results are shown below in Table 7.

TABLE 7

| | | % Inhibition |
|---|---|---|
| Sample # | Name | 0-24 hrs. |
| 1 | MeCl/su | 23 |
| 2 | MeCl/CD | 8 |
| 3 | EtAc/su | 7 |
| 4 | EtAc/CD | |
| 5 | IL-10 Control | 85 |

In this run, the MeCl/su microspheres were again the most effective during the first 24 hours.

### Results for Testing of Microspheres Formed in Third Experiment of Example 1

The microspheres formed in the third formation experiment were similarly tested for biological activity. These tests began with an IL-10 concentration of 500 ng/ml. The ELISA data obtained are shown below in Table 8.

TABLE 8

| | | IL-10 released (ng/ml) per |
|---|---|---|
| | | amount of time in culture |
| ELISA Results | | medium (DMEM) at 37° C. |
| Sample # | Name | 0-24 hrs. |
| 1 | MeCl/su A | 104 |
| 2 | MeCl/su B | 115 |
| 3 | MeCl/CD A | 280 |
| 4 | MeCl/CD B | 240 |
| 5 | Loose IL-10 | 779 |

These results show that the MeCl/CD microspheres release more IL-10 than the MeCl/su microspheres during the first 24 hours.

In this test, as in the third run above, appropriate amounts of IL-10 were taken from the supernatant of the ELISA releases and added to monocytes to achieve a final concentration of 10 ng/ml. The LM bioassay results are shown below in Table 9.

TABLE 9

| | | % Inhibition |
|---|---|---|
| Sample # | Name | 0-24 hrs. |
| 1 | MeCl/su A | 56 |
| 2 | MeCl/su B | 43 |
| 3 | MeCl/CD A | 77 |
| 4 | MeCl/CD B | 64 |
| 5 | IL-10 Control | 96 |

These results show that the MeCl/CD microspheres were more active than the MeCl/su microspheres.

### Example 4

### Testing of Pellets Formed in Example 2 for Biological Activity

The weights of the four pellets formed in Example 2 were as shown below in Table 10.

TABLE 10

| Name | Powder Weight (g) | Pellet Weight (g) |
|---|---|---|
| MeCl/su A | 0.0105 | 0.0093 |
| MeCl/su B | 0.0101 | 0.0098 |
| MeCl/CD A | 0.0098 | 0.0085 |
| MeCl/CD B | 0.0103 | 0.0091 |

The pellets were then subjected to the same ELISA and LM bioassay experiments described above in Example 3. The ELISA data were as shown below in Table 11.

TABLE 11

| | | IL-10 released (ng/ml) per |
|---|---|---|
| | | amount of time in culture |
| ELISA Results | | medium (DMEM) at 37° C. |
| Sample # | Name | 0-48 hrs. |
| 1 | MeCl/su A | 313 |
| 2 | MeCl/su B | 483 |
| 3 | MeCl/CD A | 708 |
| 4 | MeCl/CD B | 629 |
| 5 | IL-10 Control | 938 |

These results show that the CD microspheres released more IL-10 than the su microspheres.

Here, appropriate amounts of IL-10 were taken from the supernatant of the ELISA releases and added to monocytes to achieve a final concentration of 10 ng/ml. The LM bioassay results were as shown below in Table 12.

TABLE 12

| Sample | | % Inhibition |
|---|---|---|
| # | Name | 0-48 hrs. |
| 1 | MeCl/su A | 42 |
| 2 | MeCl/su B | 43 |
| 3 | MeCl/CD A | 30 |
| 4 | MeCl/CD B | 28 |
| 5 | IL-10 Control | 89 |

In this particular experiment, the MeCl/su microsphere pellets inhibited TNF-α more effectively than the MeCl/D pellets.

Both experiment 3 of Example 1 and Example 2 were repeated to create four additional pellets. The weights of the four new pellets are shown below in table 13.

TABLE 13

| Name | Powder Weight (g) | Pellet Weight (g) |
|---|---|---|
| MeCl/CD A-2 | 0.0100 | 0.0098 |
| MeCl/CD B-2 | 0.0100 | 0.0095 |
| MeCl/su A-2 | 0.0080 | 0.0073 |
| MeCl/su B-2 | 0.0105 | 0.0103 |

To test the new pellets for biological activity, each was placed in 0.5 ml of 10% IMDM (Iscove's Modified Dulbecco's Medium) in a 48 well plate at 37° C. After 24 hours, the plate was centrifuged and 50 μl of the supernatant was collected and frozen in two aliquots. The remaining pellet was washed once with phosphate buffer solution, placed in a fresh 0.5 ml of IMDM, and exposed to ultraviolet light for 30 minutes for sterilization. The process was repeated every 24 hours for 13 days, and then every week thereafter for an additional five weeks (until the pellets were no longer visible). After all the samples were collected, they were thawed and tested for biological activity in the manner described above.

The ELISA results for the new pellets were as shown below in table 14.

TABLE 14

| | | IL-10 released (ng/ml) per amount of | | | | | |
|---|---|---|---|---|---|---|---|
| | | time in culture medium (IMDM) at | | | | | |
| ELISA Results | | 37° C. | | | | | |
| Sample | | Day | Day | Day | Day | Day | Day |
| # | Name | 1 | 2 | 3 | 4 | 5 | 6 |
| 1 | MeCl/CD | 532 | 22 | 1 | 0.23 | 0.12 | 0.48 |
| | A-2 | | | | | | |
| 2 | MeCl/CD | 353 | 12 | 2 | 0.44 | 0.24 | 0.20 |
| | B-2 | | | | | | |
| 3 | MeCl/su | 202 | 5 | 0.8 | 0.20 | 0.13 | 0.10 |
| | A-2 | | | | | | |
| 4 | MeCl/su | 302 | 13 | 1.7 | 0.38 | 0.15 | 0.15 |
| | B-2 | | | | | | |

| | | IL-10 released (ng/ml) per amount of | | | | |
|---|---|---|---|---|---|---|
| ELISA Results | | time in culture medium (IMDM) at 37° C. | | | | |
| Sample | | Day | Day | Day | Day | Day |
| # | Name | 7 | 8-9 | 10 | 11 | 12 |
| 1 | MeCl/CD | 0.11 | 0.13 | 0.09 | 0.05 | 0.03 |
| | A-2 | | | | | |
| 2 | MeCl/CD | 0.14 | 0.25 | 0.14 | 0.08 | 0.04 |
| | D-2 | | | | | |
| 3 | MeCl/su | 0.06 | 0.10 | 0.07 | 0.04 | 0.03 |
| | A-2 | | | | | |
| 4 | MeCl/su | 0.09 | 0.13 | 0.11 | 0.05 | 0.03 |
| | D-2 | | | | | |

| | | IL-10 released (ng/ml) per amount of | | | | |
|---|---|---|---|---|---|---|
| ELISA Results | | time in culture medium (IMDM) at 37° C. | | | | |
| Sample | | Days | Days | Days | Days | Days |
| # | Name | 13-19 | 20-26 | 27-33 | 34-41 | 42-47 |
| 1 | MeCl/CD | 0.175 | 0.183 | 0.126 | 0.056 | 0.037 |
| | A-2 | | | | | |
| 2 | MeCl/CD | 0.244 | 0.116 | 0.176 | 0.04 | 0.029 |
| | B-2 | | | | | |
| 3 | MeCl/su | 0.127 | 0.260 | 0.146 | 0.11 | 0.023 |
| | A-2 | | | | | |
| 4 | MeCl/su | 0.190 | 0.178 | 0.148 | 0.07 | 0.04 |
| | B-2 | | | | | |

These results show an initial burst of IL-10 release (probably due to the presence of the sucrose and cyclodextrin), followed by sustained release over a period of seven weeks. The CD pellets released more IL-10 during the first 10 days, but during the final 37 days, the CD and su microspheres released at an approximately equal rate.

Appropriate amounts of IL-10 were taken from the day 1 ELISA releases and added to monocytes to achieve a final concentration of 10 ng/ml. The LM bioassay results were as shown below in table 15.

TABLE 15

| Sample | | % Inhibition |
|---|---|---|
| # | Name | 0-24 hrs. |
| 1 | MeCl/CD A-2 | 55 |
| 2 | MeCl/CD B-2 | 44 |
| 3 | MeCl/su A-2 | 35 |
| 4 | MeCl/su B-2 | 50 |
| 5 | IL-10 Control | 85 |

These results show the CD and su pellets exhibiting roughly equal inhibitory activity.

To determine the effect of the HSA and sucrose and cyclodextrin buffers, another experiment was performed. In this experiment, microspheres were prepared in the manner described in Example 1, first experiment (no HSA, no cyclodextrin or sucrose buffers), and were pressed into pellets in the manner described in Example 2. The pellets were then suspended in 0.5 ml of IMDM in a 48 well plate and kept on a rocker at 37° C. Supernatants were collected daily for the first 4 days, and then once after 8 days. After collecting the supernatants, the remaining pellet was washed once with phosphate buffer solution (0.5 ml), and the washing was collected. The ELISA results for the supernatants and the washings were as shown below in Table 16.

TABLE 16

| | | IL-10 released (ng/ml) per amount of | | | | |
|---|---|---|---|---|---|---|
| ELISA Results | | time in culture medium (IMDM) at 37° C. | | | | |
| Sample | | Day | Day | Day | Day | Days |
| # | Name | 1 | 2 | 3 | 4 | 5-8 |
| 1 | Supernate | 1.125 | 0.0724 | 0.015 | 0.012 | 0.013 |
| 2 | Washing | 0 | 0 | 0 | 0 | 0 |

These results show that without the addition of buffers, the initial burst of IL-10 release is considerably smaller. The ELISA results for the washings demonstrate that no IL-10 was lost in the washing process. Example 5

### Testing of Pellets for Biological Activity in Rats

In this experiment, the ability of IL-10 microspheres to reduce inflammation in rats is tested. First, inflammation is induced in rats using the method described in Tate et al., “Suppression of Acute and Chronic Inflammation by Dietary Gamma Linolenic Acid,” J. Rheumatology, 16:729-33 (1989). Briefly, 20 ml of sterile air is injected subcutaneously into rats to create a subcutaneous air pouch. Six days later, monosodium urate crystals are injected into the air pouches to induce chronic inflammation. Approximately 10 mg of crystals diluted in 5 ml sterile saline is injected into each air pouch.

Next, the rats are treated by implanting IL-10 microsphere pellets in the rats near the inflammation cite. The therapeutic effect of the pellets is determined by monitoring the level of swelling after 12 hours, 24 hours, and then daily. The level of swelling is measured by characterizing the level of inflammation on a 0-4 scale, as described in Tate et al. The rats show steady reduction of swelling as the pellets steadily release IL-10 over a period of at least several days.

As controls, some rats are implanted with pellets that do not contain IL-10, and some are injected with IL-10 microspheres not compressed into pellets. In addition, some rats in which inflammation is not induced are implanted with IL-10 microsphere pellets. The rats that do not receive IL-10 treatment show no significant reduction in swelling. The rats receiving microspheres not compressed into pellets show some initial reduction, but not the steady, sustained reduction experienced by the rats receiving IL-10 microsphere pellets.

### Other Embodiments

The implantable devices need not employ a drug-polymer mixture to accomplish controlled release of the drug. For example, the drug could be loaded into a device which changes shape when implanted into the body in proximity to a target site, e.g., by osmotic absorption of fluid, causing release of the drug to the target site. In addition, for the devices having a rigid exterior shell defining a hollow interior, a drug might be loaded directly into the hollow portion, without mixing the drug with a polymer. Such an embodiment might be employed, e.g., for short term release of a drug rather than long-term sustained release.

For certain types of drugs, a shaped device similar to those described above can be constructed entirely from the drug. For example, drugs such as an anti-adhesion medication might be shaped directly into an implantable device.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An implantable device for attaching tissue to a support structure inside a body and for delivering a drug to a target location near the support structure, the device comprising:
a first portion configured to engage the tissue, and a second portion configured to engage the support structure;
wherein the device includes a section that defines an internal cavity, the internal cavity having a size and shape for containing a controlled release agent comprising the drug.

2. The device of claim 1, further comprising the controlled release agent.

3. The device of claim 2, wherein the controlled release agent comprises the drug mixed with a polymer.

4. The device of claim 2, wherein the controlled release agent comprises microspheres of a polymer that contain the drug.

5. The device of claim 2, wherein the controlled release agent is configured to release the drug for a period of greater than two days.

6. The device of claim 2, wherein the controlled release agent is configured to release the drug intermittently over a period of time.

7. The device of claim 2, wherein the drug is selected from the group consisting of a down-regulatory cytokine, a pain killer, platelet derived growth factor, an antibiotic, a hormone, a prostaglandin, a protein, a peptide sequence, and a nucleic acid.

8. The device of claim 2, wherein the drug is interleukin-10.

9. The device of claim 2, wherein the drug is lidocaine.

10. The device of claim 1, wherein the second portion is configured to penetrate the tissue.

11. The device of claim 1, wherein the section has an aperture that exposes the interior cavity to bodily fluids when the device is implanted in the body.

12. The device of claim 11, further comprising a membrane that covers the aperture, wherein the membrane is permeable to bodily fluids and to the drug only when the drug is dissolved or suspended in bodily fluids.

13. The device of claim 1, wherein the support structure comprises bone.

14. The device of claim 13, wherein the device is a bone screw comprising a head and a rigid, threaded shaft, the shaft including both the second portion and the section.

15. The bone screw of claim 14, wherein the shaft defines an aperture that opens into the internal cavity.

16. The bone screw of claim 15, further comprising a membrane that covers the aperture, wherein the membrane is permeable to bodily fluids and to the drug only when the drug is dissolved or suspended in bodily fluids.

17. An implantable device for attaching tissue to a support structure inside a body and for delivering a drug to a target location near the support structure, the device comprising:
a first portion that engages the tissue, and a second portion that engages the support structure;
wherein the device includes a section formed from a material that comprises a controlled release agent comprising a drug.

18. The device of claim 17, wherein the controlled release agent comprises microspheres of a polymer that contain the drug.

19. An implantable device for delivering a drug to a desired location inside a body, the device comprising:
a rigid exterior comprising a tapered end for penetrating tissue within the body and a projection for engaging tissue within the body; and
an internal cavity in fluid communication with the rigid exterior, wherein the cavity has a size and shape for containing a controlled release agent comprising the drug.

20. The device of claim 19, wherein the rigid exterior comprises a pointed, arrow-shaped head comprising both the tapered end and the projection.

21. The device of claim 20, wherein the arrow-shaped head comprises a shaft and two projections, each projection having a first pointed end, and a second end connected to the shaft, the first ends being movable between a first position flush with the shaft, and a second position displaced away from the shaft.

22. An implantable staple for delivering a drug to a desired location within a body, the staple comprising:
at least two prongs configured to penetrate and engage tissue; and
a shaft connecting the two prongs, wherein the shaft has an internal cavity that has a size and shape for containing a controlled release agent comprising the drug.

23. The device of claim 22, wherein the shaft comprises a material degradable by bodily fluids.

24. An implantable device for delivering a drug to a target location, the device comprising:
an elongated rod curved in a generally helical shape, wherein the helical shape tapers to a point that in use penetrates soft tissue, and wherein the helical shape forms a conical interior space configured to contain a solid controlled release agent comprising the drug.

25. An implantable device for delivering a drug to a target location, the device comprising:
a body comprising a controlled release agent that includes the drug, and the body defining a through-hole for passage of a guide wire therethrough.

26. The device of claim 25, wherein the body comprises a shell that surrounds the controlled release agent.

27. The device of claim 26, wherein the shell includes a head portion and a shaft portion and defines a bore, the bore containing a medicament core that comprises the controlled release agent, wherein the medicament core defines the through-hole.

28. The implantable device of claim 27, further comprising a tissue engaging projection connected to the shaft.

29. An implantable suture anchor for delivering a drug to a desired location in a body, the suture anchor comprising:
an exterior shell defining a hole for passage of a suture therethrough;
an internal cavity within the shell and in fluid communication with the hole, wherein the cavity has a size and shape for containing a controlled release agent comprising the drug; and
a membrane covering the hole to retain the agent within the cavity, the membrane being permeable to bodily fluids and the drug only when the drug is dissolved or suspended in bodily fluids.

30. The suture anchor of claim 31, wherein the exterior shell comprises a material that is degradable by bodily fluids.

31. An implantable suture anchor for delivering a drug to a desired location within a body, the suture anchor comprising:
a pellet formed from a mixture comprising the drug and a polymer formulated for controlled release of the drug; and
a suture passing through the pellet.

32. A splaying implantable device for delivering a drug to a desired location within a body, the device comprising:
a pellet comprising a controlled release agent comprising the drug;
a splaying anchor connected to the pellet, the anchor comprising at least two prongs that in use penetrate soft tissue, wherein a distance separating the two prongs increases when the prongs are inserted into the tissue.

33. An implantable staple for delivering a drug to a desired location within a body, the staple being formed from a material comprising a mixture of the drug and a polymer, the mixture being formulated for controlled release of the drug, the staple comprising at least two prongs configured to penetrate soft tissue and a shaft connecting the two prongs.

34. An implantable device for delivering a drug to a desired location inside a body, the device comprising:
a section formed from a sheet of one or more polymer threads molded to form the section; and
an internal cavity defined by the section, the cavity having a size and shape for containing a controlled release gent comprising the drug.

35. The device of claim 34, wherein the one or more threads are woven to form the sheet.

36. The device of claim 34, wherein the one or more threads are compressed to form a mesh sheet.

37. The device of claim 34, further comprising the controlled release agent.

38. The device of claim 37, wherein the controlled release agent comprises a pellet comprising the drug.

39. A method of attaching tissue to a support structure and delivering a drug to a target location inside a body, the method comprising:
obtaining the device of claim 1; and
implanting the device within the body by engaging the second portion with the support structure, and the first portion with the tissue, whereby the agent releases the drug to the desired location over time.

40. The method of claim 39, wherein the device is made from a material degradable by bodily fluids.

41. A method of treating inflammatory disease, the method comprising:
obtaining an implantable device that in use contains a down-regulatory cytokine;
implanting the device in proximity to a site of inflammation in the body, such that the implantable device releases the down-regulatory cytokine to the site of inflammation.

42. The method of claim 41, wherein the implantable device contains a sustained release formulation that comprises the down-regulatory cytokine, such that the device releases the down regulatory cytokine steadily over a period of time greater than two days.
